(19) United States
(12) Patent Application Publication
(10) Pub. No.: US 2017/0011186 A1
(43) Pub. Date: Jan. 12, 2017

(54) SIMILAR CASE SEARCH DEVICE, SIMILAR CASE SEARCH METHOD, AND NON-TRANSITORY COMPUTER READABLE MEDIUM

(71) Applicant: FUJIFILM Corporation, Tokyo (JP)

(72) Inventor: Akira OOSAWA, Ashigarakami-gun (JP)

(73) Assignee: FUJIFILM Corporation, Tokyo (JP)

(21) Appl. No.: 15/275,820

(22) Filed: Sep. 26, 2016

Related U.S. Application Data
(63) Continuation of application No. PCT/JP2015/056371, filed on Mar. 4, 2015.

(30) Foreign Application Priority Data
Mar. 27, 2014 (JP) 2014-066287

(51) Int. Cl. G06F 19/00; A61B 6/03; A61B 5/055; G06F 17/30; G06T 7/00

(52) U.S. Cl. CPC G06F 19/345; G06F 17/3028; G06F 19/321; G06F 19/3443; G06T 7/0081; G06T 7/0012; A61B 5/055; A61B 6/032; G06T 2207/30096; G06T 2207/10004

(57) ABSTRACT

Provided is a similar case search device that can perform a similar case search in which attention is paid to the feature amounts of a plurality of regions of interest.

A feature amount calculation unit acquires the feature amount of each of a plurality of regions of interest (ROI), each of which is designated so as to include one or more different target lesions (OL) that are lesions in examination images, in examination data including one or more examination images. An individual similarity calculation unit compares the feature amount of each region of interest (ROI) with a feature amount of a case lesion (CL), which is a lesion in a case image registered in a case, and calculates an individual similarity for each region of interest (ROI). A similar case search unit searches for a similar case on the basis of a plurality of calculated individual similarities.

### CROSS-REFERENCE TO RELATED APPLICATION

This application is a Continuation of PCT International Application PCT/JP2015/056371 filed on 4 Mar. 2015, which claims priority under 35 USC 119 (a) from Japanese Patent Application No. 2014-066287 filed on 27 Mar. 2014. The above application is hereby expressly incorporated by reference, in its entirety, into the present application.

### BACKGROUND OF THE INVENTION

1. Field of the Invention

The present invention relates to a similar case search device, a similar case search method, and a non-transitory computer readable medium.

2. Description of the Related Art

In the medical field, a similar case search device has been known which searches for a past case that is similar to an examination image on the basis of the examination image (for example, see JP2010-237930A and JP2012-118583A (US2012/134555A)). The examination image is, for example, an image captured by a modality, such as a computed tomography (CT) apparatus that performs tomography or a general X-ray apparatus that captures a simple transparent image, and is used to diagnose a patient, for example, to specify the disease of a patient. In some cases, in one examination operation using the general X-ray apparatus, only one examination image is captured or a plurality of examination images are captured. In one examination operation using the CT apparatus, a plurality of tomographic images (slice images) are acquired. Therefore, one examination data item includes one or more examination images. In many cases, the past examination data is accumulated to create a case. Therefore, data of one case includes one or more case images.

In a case in which a similar case search is performed, first, a user, such as a doctor, designates a region of interest in an examination image. The region of interest indicates a region in which the doctor is particularly interested in the examination image and which includes a lesion to be diagnosed. The similar case search device compares a feature amount which is obtained by quantifying the features of one region of interest designated in the examination image and a feature amount which is obtained by quantifying the features of one lesion in a case image and determines the similarity therebetween. Here, for convenience of explanation, a lesion which is included in the region of interest of the examination image is referred to as a target lesion and a lesion which is included in the case image is referred to as a case lesion. Then, the similar case search device searches for a case including a case lesion that is similar to the region of interest from a case database storing a plurality of cases.

In general, the users designate the region of interest including a target lesion, using different methods, and a variation in search, that is, a variation in the search result occurs due to the difference between individuals. JP2010-237930A discloses a technique which reduces the variation in search. Specifically, even in a case in which a region including the same target lesion is designated as the region of interest, the shape or size of the designated region varies due to the difference in how the user designates the region of interest. As a result, a feature amount is likely to be changed. In the event that the feature amount is changed, similarity is also changed, which results in a variation in search in which the search result varies depending on the user. In JP2010-237930A, in order to reduce the variation in search, for example, the feature amount of each of a plurality of regions of interest in which one target lesion is designated by different methods is calculated, similarity is calculated on the basis of the average value of the calculated feature amounts of the plurality of regions of interest, and a similar image is searched. According to this structure, it is possible to reduce a variation in search due to the difference in designation between the users.

JP2012-118583A (US2012/134555A) relates to a technique that outputs the search result which is more suitable than the subjective feeling of the user on similarity. Specifically, in a case in which the same type of target lesion is present in a plurality of examination images, in the event that a region of interest is designated, the regions of interest including a plurality of target lesions of the same type which the user feels to be similar to each other are put into one group as a group of the same type of target lesions. In one examination data item, a feature amount range including all of the feature amounts of a plurality of target lesions belonging to the group of the same type of target lesions is calculated and a similar case search is performed, using the feature amount range as a search condition. Since it is considered that the feature amount range of the group of the same type of target lesions is equal to that of the target lesions which the user subjectively feels to be similar to each other, the search result is more suitable than the subjective feeling of the user.

However, in some cases, a plurality of target lesions appear in an examination image depending on a disease, which is a basis for specifying a disease. For example, in the case of tuberculosis, a disease is specified on the basis of three types of target lesions, that is, a vomica shadow (cavity), a punctate shadow (small nodules), and a frosted glass shadow (ground glass opacity), which appear in an examination image. In the case of diffuse panbronchiolitis, a disease is specified on the basis of two types of target lesions, that is, an abnormal shadow of the bronchus and a punctate shadow. In the case of a cancer, a case that is similar to a single target lesion may be searched. In the case of non-cancerous diseases other than cancer, it is necessary to search for a case that is similar to a plurality of target lesions.

In the similar case search devices disclosed in JP2010-237930A and JP2012-118583A (US2012/134555A), attention is paid to one target lesion included in the examination image and a similar case is searched on the basis of the feature amount of the region of interest including one target lesion to which attention is paid. However, it is not considered that attention is paid to each of a plurality of target lesions included in the examination images.

As described above, in JP2010-237930, the feature amount is calculated for each region of interest. A plurality of regions of interest are designated by different methods, but have the same target lesion. Therefore, JP2010-237930 does not disclose a technique that pays attention to the feature amounts of a plurality of regions of interest including different target lesions and searches for a similar case. In addition, in JP2012-118583A (US2012/134555A), for a plurality of target lesions included in a plurality of examination images, one search condition is created for one group of the same type of target lesions and a similar case is searched under the created search condition. In other words, in JP2012-118583A (US2012/134555A), the feature amount common to the regions of interest including a plurality of target lesions of the same type is calculated according to the user's preference. However, JP2012-118583A (US2012/134555A) does not disclose a technique that pays attention to the feature amounts of a plurality of regions of interest including a plurality of target lesions and searches for a similar case.

### SUMMARY OF THE INVENTION

An object of the invention is to provide a similar case search device and a similar case search method that can perform a similar case search in which attention is paid to the feature amounts of a plurality of regions of interest, and a non-transitory computer readable medium.

A similar case search device according to the invention searches for a similar case which is similar to an examination image used to diagnose a patient from a case database in which a plurality of cases, each of which includes one or more case images, are registered. The similar case search device comprises a feature amount acquisition unit, an individual similarity calculation unit, and a similar case search unit. The feature amount acquisition unit acquires a feature amount of each of a plurality of regions of interest, each of which is designated so as to include one or more different target lesions that are lesions in the examination images, in examination data including one or more examination images. The individual similarity calculation unit compares the feature amount of each region of interest with a feature amount of a case lesion, which is a lesion in the case image registered in the case, and calculates an individual similarity for each region of interest. The similar case search unit searches for the similar case on the basis of a plurality of calculated individual similarities.

The plurality of regions of interest may include different types of lesions. In a case in which a plurality of case lesions are registered in one case, preferably, the individual similarity calculation unit sets the plurality of regions of interest and the plurality of case lesions so as to be in one-to-one correspondence with each other, compares the feature amounts, and calculates an individual similarity for each case lesion.

Here, the case in which a plurality of case lesions are registered in one case, that is, a plurality of case lesions are present in the case images includes a case in which a plurality of case lesions are present in one case image and a case in which the sum of the case lesions that are present in a plurality of case images is two or more, for example, a case in which one case lesion is present in each of two case images.

Preferably, the similar case search unit creates a similar case list which is a list of information related to a plurality of similar cases. Preferably, the similar case list includes a similar case list for each region of interest.

Preferably, the similar case list for each region of interest is a list of the plurality of case lesions. In a case in which there are a plurality of case lesions included in a common case in each similar case list for each region of interest, preferably, the plurality of case lesions included in the common case are identifiably displayed to indicate that the case lesions are included in the common case. Preferably, the similar case search unit sorts the plurality of case lesions on the basis of the individual similarity in the similar case list.

Preferably, the similar case search device further includes a representative value determination unit that, in a case in which the individual similarities are calculated between one region of interest and a plurality of case lesions included in one case, determines one representative value from a plurality of individual similarities. Preferably, the similar case search unit searches for the similar case for one region of interest, using only the case lesion corresponding to the representative value.

Preferably, the individual similarity calculation unit calculates the individual similarity, using a correspondence between one region of interest and a case lesion that is the same type as the region of interest among the case lesions included in one case. Preferably, the similar case search device further includes a lesion type determination unit that determines the type of lesion on the basis of the feature amount of the region of interest.

A similar case search method according to the invention searches for a similar case which is similar to an examination image used to diagnose a patient from a case database in which a plurality of cases, each of which includes one or more case images, are registered. The similar case search method comprises a feature amount acquisition step, an individual similarity calculation step, and a similar case search step. In the feature amount acquisition step, a feature amount of each of a plurality of regions of interest, each of which is designated so as to include one or more different target lesions that are lesions in the examination images, in examination data including one or more examination images is acquired. In the individual similarity calculation step, the feature amount of each region of interest is compared with a feature amount of a case lesion, which is a lesion in the case image registered in the case, and an individual similarity for each region of interest is calculated. In the similar case search step, the similar case is searched on the basis of a plurality of calculated individual similarities.

A non-transitory computer readable medium according to the invention stores a computer-executable program enabling execution of computer instructions to perform operations for searching for a similar case which is similar to an examination image used to diagnose a patient from a case database in which a plurality of cases, each of which includes one or more case images, are registered. The operations include acquiring a feature amount of each of a plurality of regions of interest, each of which is designated so as to include one or more different target lesions that are lesions in the examination images, in examination data including one or more examination images, comparing the feature amount of each region of interest with a feature amount of a case lesion, which is a lesion in the case image registered in the case, and calculating an individual similarity for each region of interest, and searching for the similar case on the basis of a plurality of calculated individual similarities.

The feature amounts of a plurality of regions of interest are compared with the feature amounts of each case lesion included in the case images to calculate the individual similarities and a similar case is searched on the basis of the calculated individual similarities. Therefore, it is possible to simply perform a similar case search in which attention is paid to the feature amounts of a plurality of regions of interest.

### BRIEF DESCRIPTION OF THE DRAWINGS

FIG. 1 is a diagram illustrating the structure of a medical information system including a similar case search server.

FIG. 2 is a diagram schematically illustrating examination data including a plurality of examination images.

FIG. 3 is a diagram schematically examination data including one examination image.

FIG. 4 is a diagram illustrating the functions of a treatment department, an examination department, an examination image DB server, and a case DB server.

FIG. 5 is a diagram illustrating a case DB.

FIG. 6 is a diagram illustrating the outline of the functions of a similar case search server.

FIG. 7 is a diagram illustrating the structure of a computer forming each DB server or each terminal.

FIG. 8 is a diagram schematically illustrating the structure of a treatment department terminal.

FIG. 9 is a diagram illustrating an examination image display screen for designating a region of interest.

FIG. 10 is a diagram illustrating an example of a method for designating the region of interest which is different from that illustrated in FIG. 9.

FIG. 11 is a diagram schematically illustrating the structure of the similar case search server.

FIG. 12 is a diagram illustrating the feature amount of a region of interest.

FIG. 13 is a diagram illustrating lesion image patterns.

FIG. 14 is a diagram illustrating the structure of a feature amount calculation unit.

FIG. 15 is a diagram illustrating the feature amount of a case lesion.

FIG. 16 is a diagram illustrating an individual similarity calculation unit.

FIG. 17 is a diagram illustrating an individual similarity calculation method.

FIG. 18 is a diagram illustrating individual similarities corresponding to a plurality of regions of interest.

FIG. 19 is a diagram illustrating individual similarities which are calculated from one region of interest and a plurality of case lesions.

FIG. 20 is a diagram illustrating an ISM table.

FIG. 21 is a diagram schematically illustrating the ISM tables created for each region of interest.

FIG. 22 is a diagram illustrating a TSM table creation method.

FIG. 23 is a diagram illustrating a screen on which a similar case list is displayed.

FIG. 24 is a diagram illustrating a screen on which lists of similar cases for each region of interest are separately displayed.

FIG. 25 is a flowchart illustrating the outline of a process of a similar case search device.

FIG. 26 is a flowchart illustrating the procedure of a process in the similar case search server.

FIG. 27 is a diagram illustrating an average rank list according to a second embodiment.

FIG. 28 is a diagram illustrating a modification example of the second embodiment.

FIG. 29 is a diagram illustrating a representative value determination unit according to a third embodiment.

FIG. 30 is a diagram illustrating a determination method according to the third embodiment.

FIG. 31 is a diagram illustrating an ISM table according to the third embodiment.

FIG. 32 is a flowchart illustrating a process according to the third embodiment.

FIG. 33 is a diagram illustrating a fourth embodiment.

FIG. 34 is a diagram illustrating a determination method according to a fourth embodiment.

FIG. 35 is a diagram illustrating a case DB according to the fourth embodiment.

FIG. 36 is a diagram illustrating an individual similarity calculation method according to the fourth embodiment.

FIG. 37 is a diagram illustrating a fifth embodiment.

FIG. 38 is a diagram illustrating a similar case search request according to the fifth embodiment.

### DESCRIPTION OF THE PREFERRED EMBODIMENTS

### First Embodiment

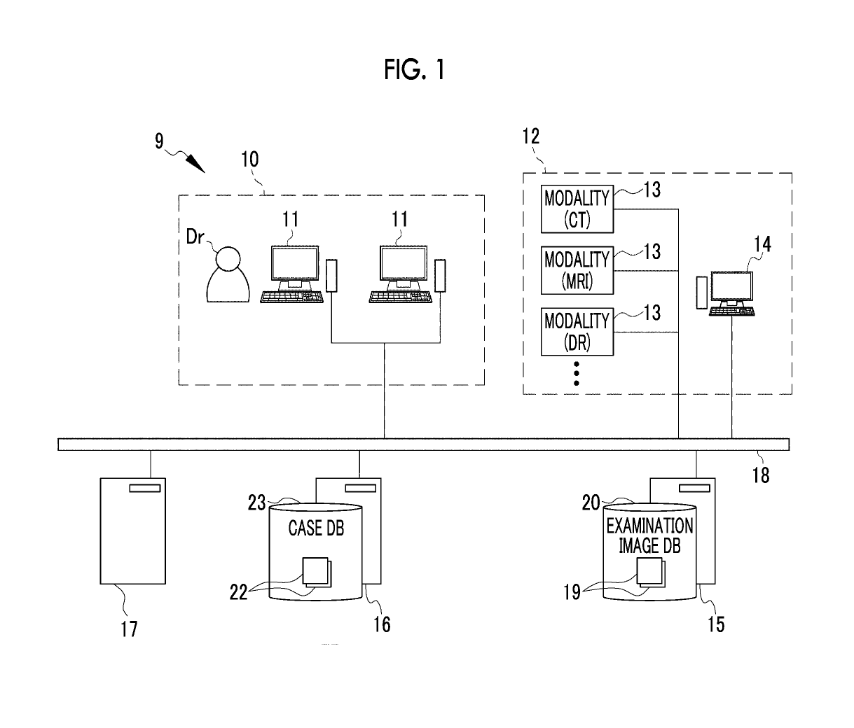

A medical information system 9 illustrated in FIG. 1 is constructed in a medical facility such as a hospital. The medical information system 9 includes a treatment department terminal 11 that is provided in a treatment department 10, a modality (medical imaging apparatus) 13 and an order management terminal 14 that are provided in an examination department 12, an examination image database (hereinafter, referred to as a “DB”) server 15, a case DB server 16, and a similar case search server 17. These components are connected through a network 18 such that they can communicate with each other. The network 18 is, for example, a local area network (LAN) which is constructed in a hospital. The modality 13 includes a computed tomography (CT) apparatus or a magnetic resonance imaging (MRI) apparatus that captures tomographic images and a general X-ray apparatus (for example, digital radiography (DR) or computed radiography (CR)) that captures simple transparent images.

The treatment department terminal 11 is operated by a doctor (to which letters “Dr” are attached in the drawings) in the treatment department 10 to input or browse electronic medical records and to issue an examination order for examination to the examination department 12. In addition, the treatment department terminal 11 is used as an image display terminal that displays an examination image 19 which has been captured in the examination department 12 and then stored in the examination image DB server 15 such that the doctor can browse the examination image 19.

In the examination department 12, the order management terminal 14 receives the examination order from the treatment department 10 and manages the received examination order. A technician in the examination department 12 takes a radiographic image of a patient using the modality 13 according to the content of the examination order. One or a plurality of examination images 19 are captured in response to one examination order. In the event that imaging ends, the modality 13 transmits the captured examination image 19 to the examination image DB server 15. In the event that examination ends, the doctor in the treatment department 10 is notified of the end of the examination from the examination department 12 and is also notified of the storage destination of the examination image 19 in the examination image DB server 15. The doctor in the treatment department 10 accesses the examination image DB server 15 through the treatment department terminal 11 and browses the examination image 19 using the treatment department terminal 11.

The examination image DB server 15 includes an examination image DB 20 that stores a plurality of examination images 19 and is a so-called picture archiving and communication system (PACS) server. The examination image DB 20 is a database which can be searched by keyword and transmits an examination image 19 matched with search conditions or a designated examination image 19 in response to, for example, a search request or a transmission request from the treatment department terminal 11.

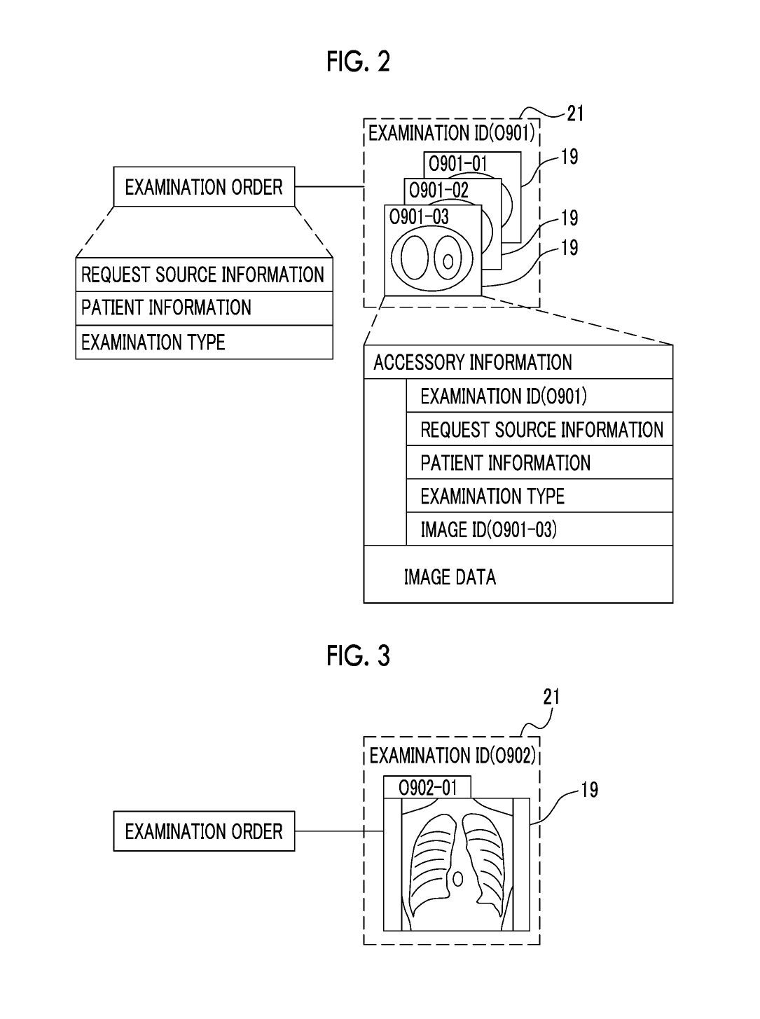

As illustrated in FIGS. 2 and 3, in the examination image DB 20, one examination data item 21 including one or more examination images 19 is stored so as to be associated with one examination order. As illustrated in FIG. 2, the examination image 19 captured by the CT apparatus or the MRI apparatus is a tomographic image (also referred to as a slice image) and one examination data item 21 includes a plurality of examination images 19. As illustrated in FIG. 3, the examination image 19 captured by the general X-ray apparatus is a simple transparent image. One examination data item 21 may include only one examination image 19 or a plurality of examination images 19.

The examination order includes, for example, information about a request source, such as the ID (identification data) or position of the doctor in the treatment department 10, patient information, and the type of examination. An image file of the examination image 19 includes image data and accessory information such as a digital imaging and communication in medicine (DICOM) header. Examination order information is stored as the accessory information of the examination image 19. In addition, the accessory information includes an examination ID and an image ID which is given to each examination image 19. In the example illustrated in FIGS. 2 and 3, the examination ID is “O901” or “O902” and the image ID is given in the form in which a serial number for identifying one examination image 19 is added to the examination ID. For example, the image ID is “O901-03” or “O901-01”. The examination image DB server 15 can perform a search, using an item included in a DICOM tag as a search key.

The similar case search server 17 receives the examination image 19 as search conditions and searches for a case including a case image 22 that is similar to the received examination image 19. The case image 22 is an examination image that was used for diagnosis in the past. The case DB server 16 includes a case DB 23 that stores a plurality of cases such that the cases can be searched. The similar case search server 17 accesses the case DB server 16, reads out the cases one by one, compares the examination image 19 which has been received as the search conditions with the case image 22 in the case, and searches for a case that is similar to the examination image 19.

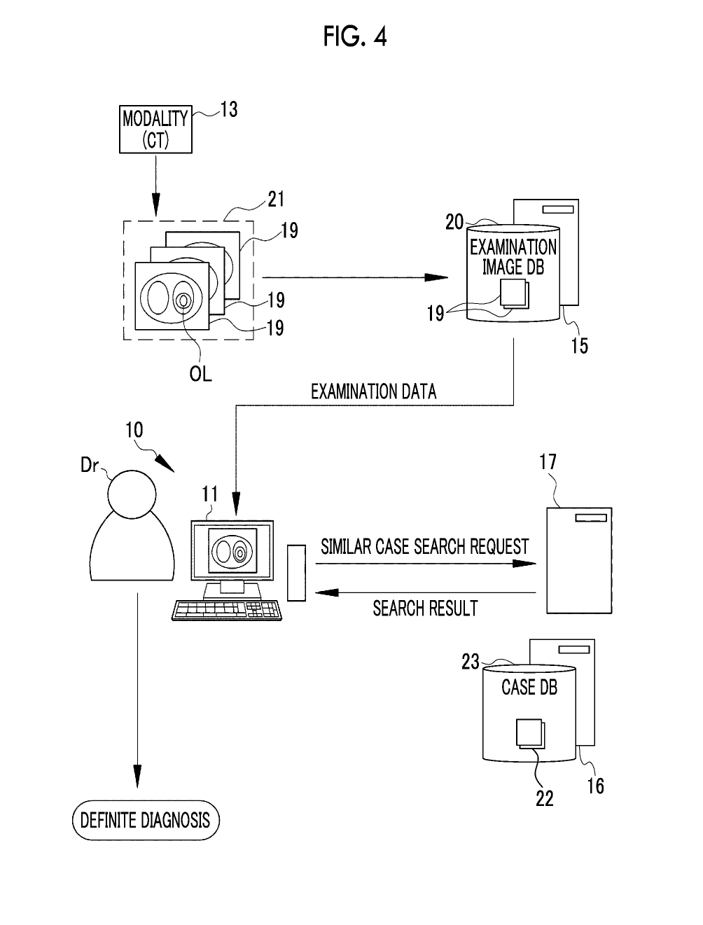

As illustrated in FIG. 4, the doctor in the treatment department 10 accesses the examination image DB server 15 and downloads examination data 21 including the examination images 19, using the treatment department terminal 11. The examination image 19 is displayed on the treatment department terminal 11 and is browsed by the doctor. In a case in which a patient has a disease, a lesion (also referred to as a target lesion OL) indicating the symptoms of the disease is included in the examination image 19 of the patient. The doctor in the treatment department 10 selects an examination image 19 including the target lesion OL from the examination images 19 included in the examination data 21. The examination image 19 is added to a similar case search request that is issued from the treatment department terminal 11 to the similar case search server 17 and the similar case search request is transmitted to the similar case search server 17. In the event of receiving the similar case search request, the similar case search server 17 searches for a case similar to the examination image 19 from the case DB server 16 and transmits the search result to the treatment department terminal 11 which is a request source.

The doctor in the treatment department 10 checks the case included in the examination result. The case includes a radiogram interpretation report associated with the case image 22. The doctor makes a definite diagnosis, such as the specification of a disease in the examination image 19, with reference to, for example, an opinion on the case image 22 which is written in the radiogram interpretation report.

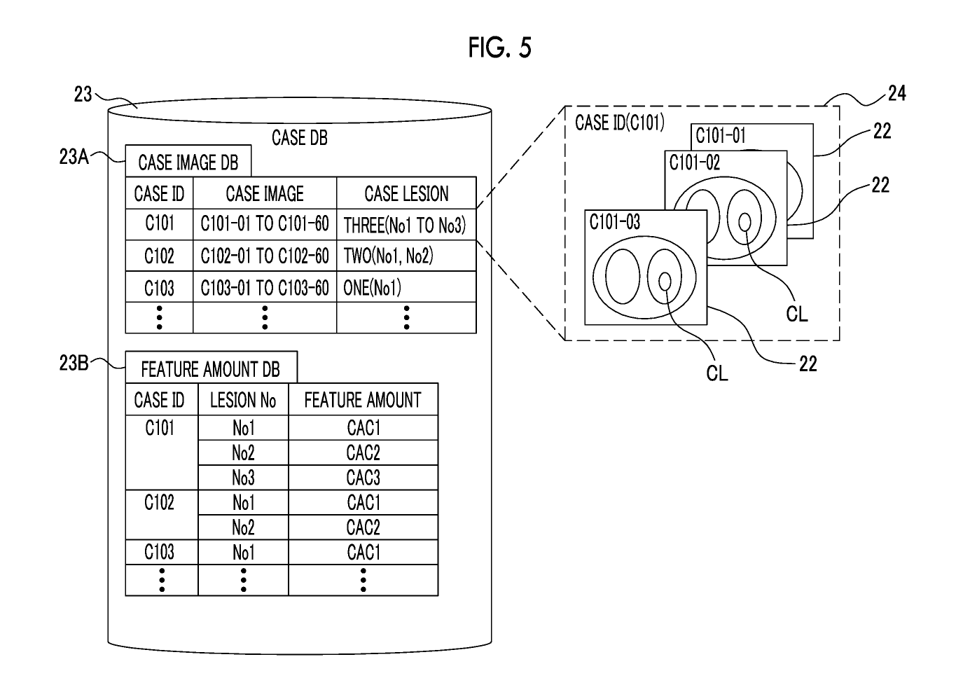

As illustrated in FIG. 5, the case DB 23 includes a case image DB 23A and a feature amount DB 23B. The case image DB 23A is a database which stores the case image 22 such that the case image 22 can be searched. A case ID is given to each case. The case ID corresponds to the examination ID in the examination image 19. One case includes one or more case images 22. Similarly to the examination image 19, an image ID in which a serial number is added to the case ID is given to each case image 22. In FIG. 5, case data 24 with a case ID “C101” includes, for example, 60 tomographic images.

The case image 22 includes a lesion (case lesion CL) indicating the symptoms of a disease. One or more case lesions CL are registered in one case. In this example, three case lesions CL with No1 to No3 are registered in a case with a case ID “C101”, two case lesions CL are registered in a case with a case ID “C102”, and one case lesion CL is registered in a case with a case ID “C103”. The case lesion CL is a region that was designated as a lesion by the doctor in the event that the case image 22 was used as the examination image for diagnosis in the past and was registered as the case lesion CL by the doctor through a definite diagnosis. A method for designating the case lesion CL is the same as, for example, a method for designating a region of interest ROI which will be described below.

The feature amount DB 23B is a database that stores the feature amount CAC of an image of the case lesion CL. An ID including the case ID and a lesion number (No) is given to the feature amount CAC. For example, there are three case lesions CL in the case ID “C101” and serial numbers No1 to No3 in one case are given to each case lesion CL. A number following the feature amount CAC corresponds to the serial number in the case. A method for calculating the feature amount CAC is the same as, for example, a method for calculating the region of interest ROI which will be described below.

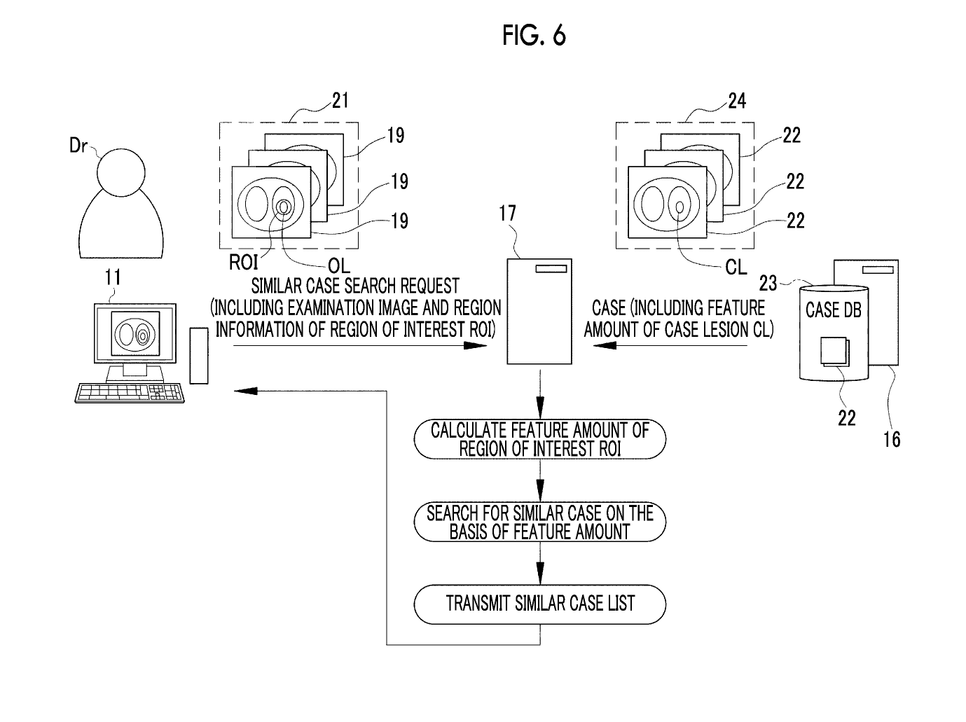

As illustrated in FIG. 6, in the event that the similar case search request is issued through the treatment department terminal 11, the doctor designates a region including the target lesion OL in the examination image 19 as the region of interest ROI. The examination image 19 including the target lesion OL and information about a region which corresponds to the designated region of interest ROI in the examination image 19 (for example, information about coordinates in the examination image 19) are added to the similar case search request. In the event of receiving the similar case search request, the similar case search server 17 specifies the region of interest ROI on the basis of image data of the examination image 19 and the region information. Then, the similar case search server 17 calculates the feature amount of the region of interest ROI. Then, the similar case search server 17 reads out the cases one by one from the case DE server 16, compares the feature amounts of the region of interest ROI and the case lesion CL, and searches for similar cases. Then, the similar case search server 17 creates a similar case list which is a list of information related to a plurality of similar cases and transmits the similar case list as the search result to the treatment department terminal 11.

The treatment department terminal 11, the order management terminal 14, the examination image DB server 15, the case DB server 16, and the similar case search server 17 are implemented by installing a control program, such as an operating system, or an application program, such as a client program or a server program, in computers, such as personal computers, server computers, or workstations.

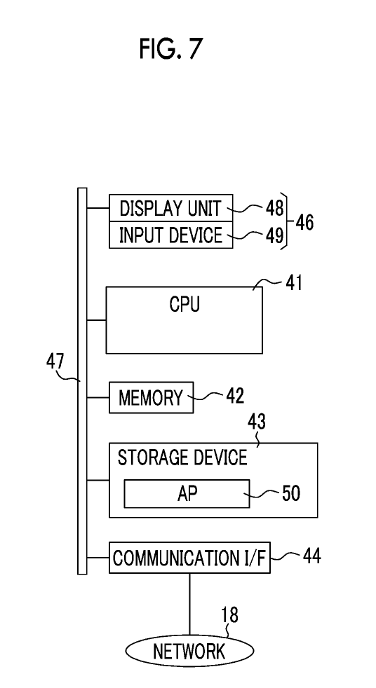

As illustrated in FIG. 7, the computers forming the DE servers 15 to 17 and the terminals 11 and 14 have the same basic structure and each comprise a central processing unit (CPU) 41, a memory 42, a storage device 43, a communication I/F 44, and an input/output unit 46. These components are connected to each other through a data bus 47. The input/output unit 46 includes a display unit 48 and an input device 49 such as a keyboard or a mouse.

The storage device 43 is, for example, a hard disk drive (HDD) and stores a control program or an application program (hereinafter, referred to as an AP) 50. In addition to the HDD storing the programs, a disk array obtained by connecting a plurality of HDDs is provided as the storage device 43 for a DB in a server in which a DB is constructed. The disk array may be provided in the main body of the server, or it may be provided separately from the main body of the server and may be connected to the main body of the server through a cable or a network.

The memory 42 is a work memory that is used by the CPU 41 to perform processes. The CPU 41 loads the control program stored in the storage device 43 to the memory 42 and performs a process according to the program to control the overall operation of each unit of the computer. The communication I/F 44 is a network interface that controls communication with the network 18.

As the AP 50, a client program, such as electronic medical record software for browsing or editing electronic medical records or viewer software for browsing examination images or a similar case list, is installed in the treatment department terminal 11. The viewer software may be, for example, dedicated software or a general-purpose web browser.

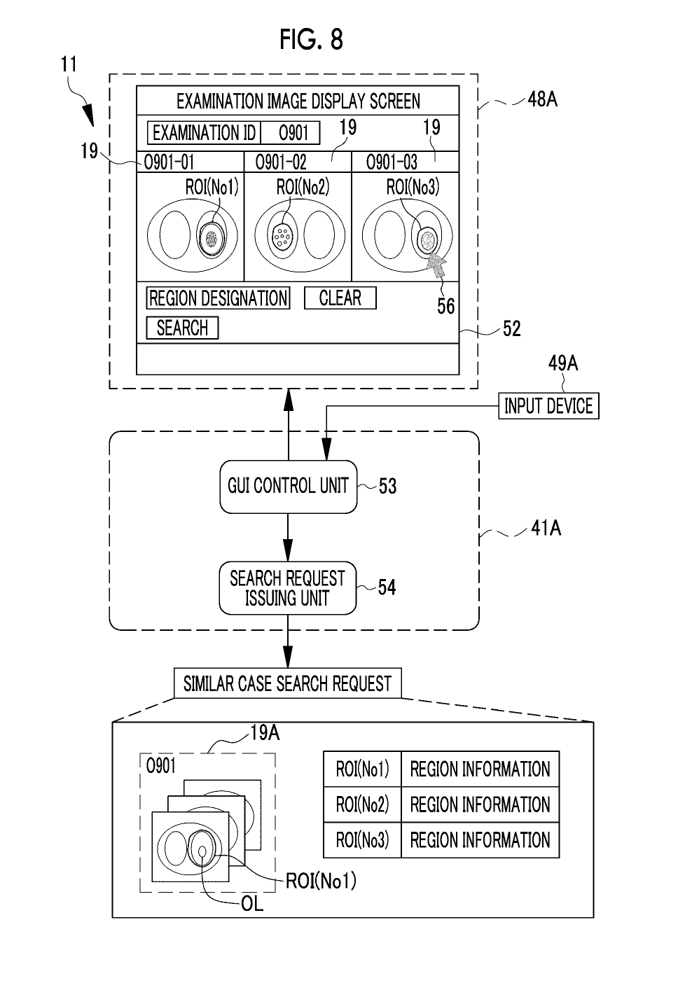

As illustrated in FIG. 8, in the treatment department terminal 11, in the event that viewer software for displaying the examination images 19 starts, an examination image display screen 52 having an operation function by a graphical user interface

(GUI) is displayed on a display unit 48A of the treatment department terminal 11. A CPU 41A of the treatment department terminal 11 functions as a GUI control unit 53 and a search request issuing unit 54. An operation of designating the region of interest ROI in the examination image 19 and an operation of instructing the issue of a similar case search request can be performed through the examination image display screen 52. The GUI control unit 53 receives an operation instruction from an input device 49A through the examination image display screen 52 and performs screen control corresponding to the received operation instruction. In the event that an instruction to issue a similar case search request is input, the input issuing instruction is input from the GUI control unit 53 to the search request issuing unit 54. The search request issuing unit 54 adds the designated examination image 19 or the region information of the designated region of interest ROI to the similar case search request and issues the similar case search request.

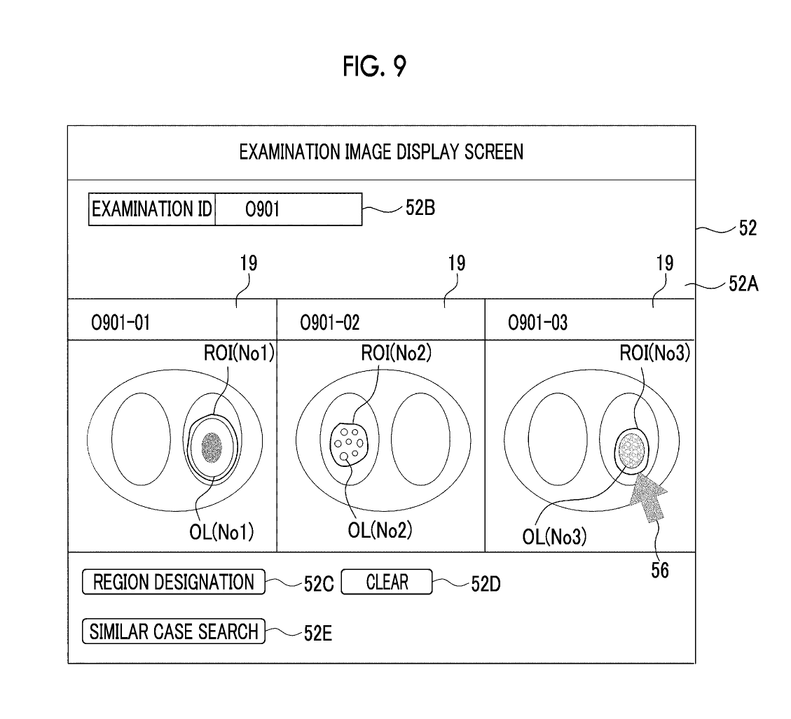

As illustrated in FIG. 9, the examination image display screen 52 includes an image display region 52A in which the examination image 19 is displayed and various operation portions. For example, three examination images 19 are displayed side by side in the image display region 52A. The examination images 19 to be displayed can be switched by a scroll operation or a frame advance operation. An input box 52B for inputting an examination ID is provided in an upper part of the image display region 52A. In the event that an examination ID is input to the input box 52B, examination data 21 with the input examination ID is downloaded from the examination image DB server 15 and the examination image 19 is displayed in the image display region 52A. A region designation button 52C, a clear button 52D, and a similar case search button 52E are provided below the image display region 52A.

The region designation button 52C is an operation button for designating the region of interest ROI in the examination image 19. In the event that the region designation button 52C is clicked by a pointer 56 of a mouse, a region designation operation which designates an arbitrary region of the examination image 19 can be performed. In this state, the pointer 56 is operated to designate the outer circumference of a region including a target lesion OL, using, for example, a spline. The spline is a smooth curve that passes through a plurality of designated control points and is input by the designation of the control points by the pointer 56. The region including the target lesion OL is designated as the region of interest ROI by the above-mentioned operation. The clear button 52D is a button for clearing the designated region of interest ROI.

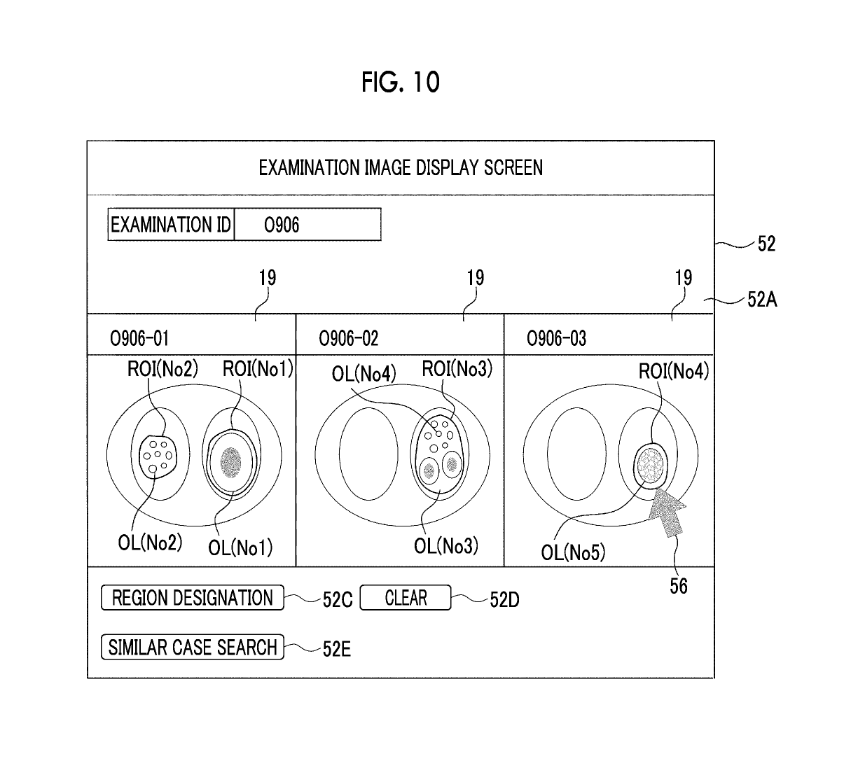

A plurality of regions of interest ROI can be designated. In the example illustrated in FIG. 9, the regions of interest ROI with No1 to No3 are designated in three examination images 19 with image IDs “O901-01” to “O901-03”, respectively. In examination data 21 with an examination ID “901”, in the event that no other regions of interest ROI are designated, a total of three regions of interest ROI are designated in one examination data item 21. In the example illustrated in FIG. 10, two regions of interest ROI (No1 and No2) are designated in an examination image 19 with an image ID “O906-01” and the regions of interest ROI (No3 and No4) are designated in examination images 19 with image IDs and “O906-02” and “O906-03”, respectively. The region of interest ROI with No3 includes two target lesions OL (No3 and No4). As such, a region including a plurality of target lesions OL may be designated as one region of interest ROI. In examination data 21 with an examination ID “906”, in the event that no other regions of interest ROI are designated, a total of four regions of interest ROI are designated in one examination data item 21. Each of the designated regions of interest ROI includes one or more different target lesions OL.

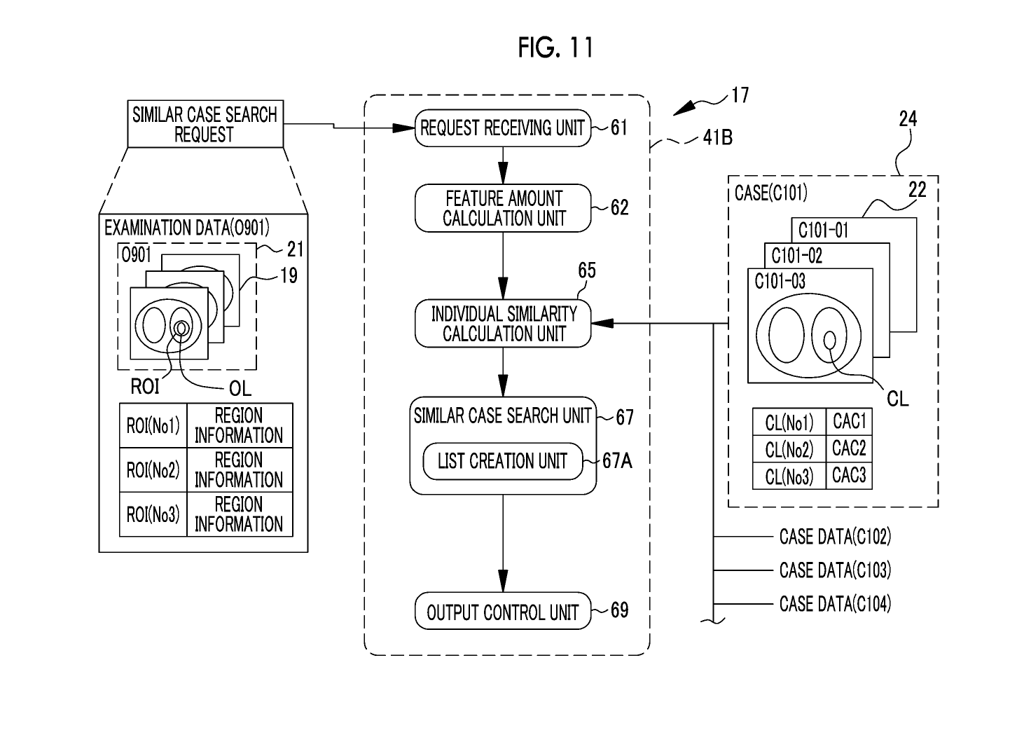

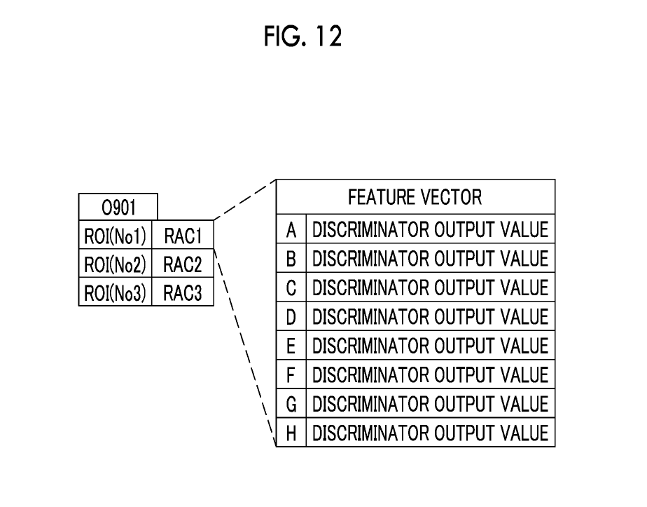

As illustrated in FIG. 11, a similar case search server program is installed as the AP 50 in the similar case search server 17. In the event that the program is executed, a CPU 41B of the similar case search server 17 functions as a request receiving unit 61, a feature amount calculation unit 62, an individual similarity calculation unit 65, a similar case search unit 67, and an output control unit 69.

The request receiving unit 61 receives the similar case search request transmitted from the treatment department terminal 11 and stores the received examination image 19 and the received region information of the region of interest ROI in, for example, the storage device 43 of the similar case search server 17. The feature amount calculation unit 62 calculates the feature amount of the region of interest ROI on the basis of the received examination image 19 and region information. Here, the feature amount calculation unit 62 functions as a feature amount acquisition unit.

As illustrated in FIG. 12, in a case in which there are a plurality of regions of interest ROI, the feature amounts RAC of the regions of interest ROI are calculated for each region of interest ROI. For example, the feature amount of a region of interest ROI with Not is “RAC1”, the feature amount of a region of interest ROI with No2 is “RAC2”, and the feature amount of a region of interest ROI with No3 is “RAC3”. The feature amount RAC is a feature vector formed by multi-dimensional values (discriminator output values which will be described below) corresponding to a plurality of types of lesion patterns which are preset as the image patterns of the typical lesions.

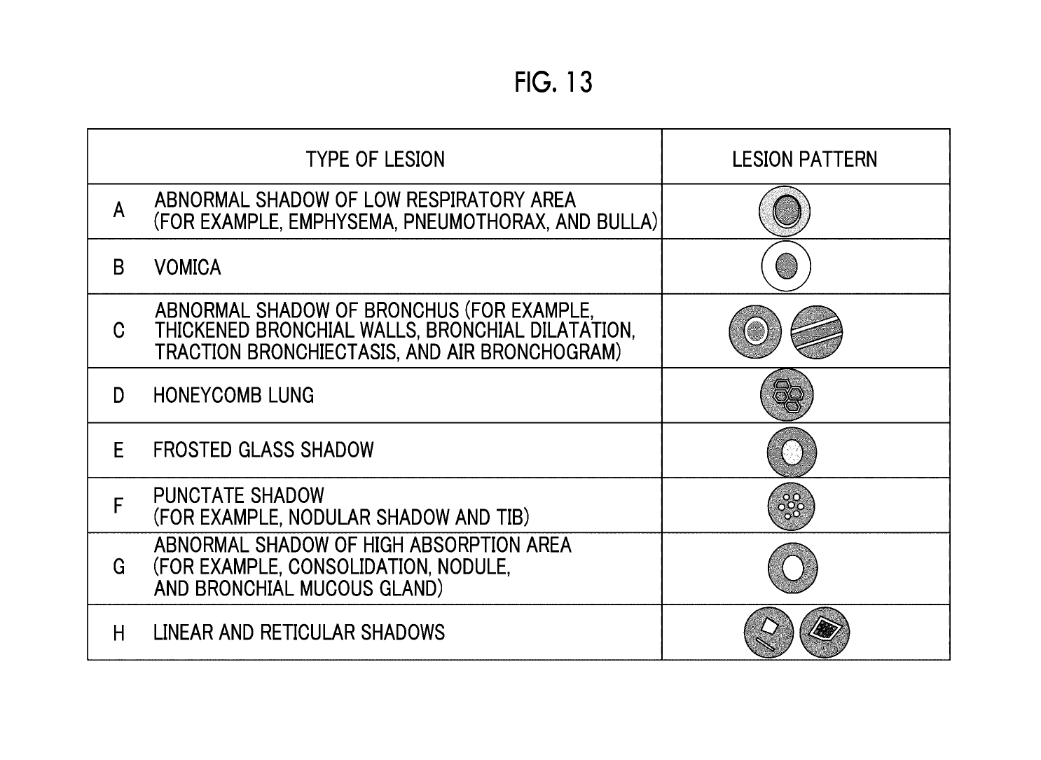

As illustrated in FIG. 13, the typical lesion patterns are classified into eight types, that is, A: an abnormal shadow of a low respiratory area (low attenuation area, such as emphysema, pneumothorax, or bulla), B: vomica, C: an abnormal shadow of the bronchus (such as thickened bronchial walls, bronchial dilatation, traction bronchiectasis, or air bronchogram), ID: a honeycomb lung (honeycombing), E: a frosted glass shadow (ground glass opacity), F: a punctate shadow (small nodules, such as a nodular shadow or TIB), G: an abnormal shadow of a high absorption area (high attenuation area, such as consolidation, nodule, or bronchial mucous gland (mucoid impaction)), and H: linear and reticular shadows.

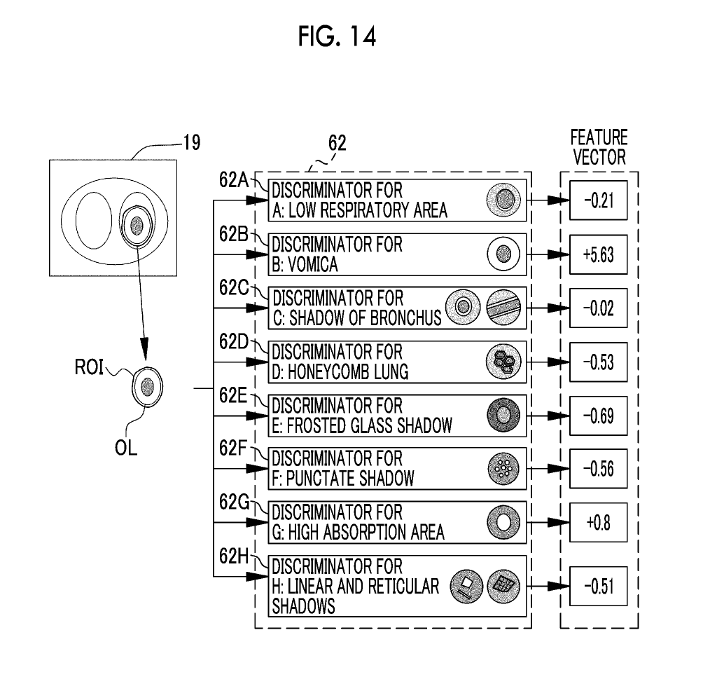

As illustrated in FIG. 14, the feature amount calculation unit 62 includes discriminators 62A to 62H corresponding to eight types of typical lesion patterns. Each of the discriminators 62A to 62H outputs values corresponding to each of the typical lesion patterns on the basis of the image pattern of the region of interest ROI. Each of the values output from the discriminators 62A to 62H is multi-dimensional numerical values forming the feature vector. Here, each value is referred to as a discriminator output value. In this example, there are eight types of discriminator output values corresponding to the discriminators 62A to 62H and a feature vector is an eight-dimensional feature vector. In this example, there are eight types of typical lesion patterns A to H. However, the number of types is less than 8 or equal to or greater than 8. The type of discriminator and the number of dimensions of the feature vector are appropriately determined on the basis of the type.

The discriminator output value indicates the likeness of the typical lesion pattern and indicates the probability of the typical lesion pattern being present in the region of interest ROI. Therefore, as the discriminator output value increases, the probability of the typical lesion pattern being present in the region of interest ROI increases. As the discriminator output value decreases, the probability of the typical lesion pattern being present in the region of interest ROI decreases. Specifically, a “positive (+)” discriminator output value indicates that the typical lesion pattern is present in the region of interest ROI and a “negative (−)” discriminator output value indicates that no typical lesion pattern is present in the region of interest ROI. In the event that the discriminator output value is “positive (+)” and becomes larger, the probability of the typical lesion pattern being present becomes higher.

As can be seen from the example illustrated in FIG. 14, the discriminator 62B corresponding to the lesion pattern “B: vomica” and the discriminator 62G corresponding to the lesion pattern “G: high absorption area” output a “+” value and the output value from the discriminator 62B corresponding to the lesion pattern “B: vomica” is the largest. Therefore, the region of interest ROI includes the lesion pattern “B: vomica” and the lesion pattern “G: high absorption area” and “B: vomica” among the eight types of lesion patterns has a dominant image pattern.

Each of the discriminators corresponding to the typical lesion patterns can be created by a machine learning algorithm, such as “Ada-boost”, using, for example, a well-known feature amount described in “Document Name: Computer Vision and Image Understanding, vol. 88, pp. 119 to 151, December 2002, and Chi-Ren Shyu, Christina Pavlopoulou Avinash C. kak, and Cala E. Brodley, “Using Human Perceptual Categories for Content-Based Retrieval from a Medical Image Database”.

The feature amount calculation unit 62 calculates the feature amount RAC of each of a plurality of regions of interest ROI designated in the examination data 21 attached to the similar case search request.

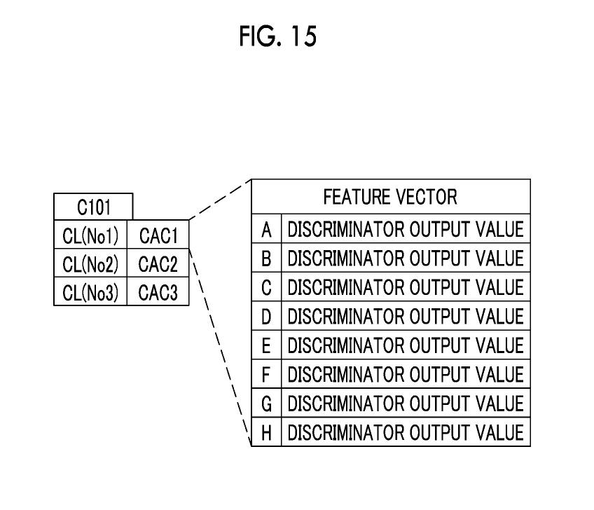

As illustrated in FIG. 15, the feature amount CAC of each case lesion CL stored in a feature amount DB 23B of the case DB 23 is formed by a feature vector corresponding to the eight types of lesion patterns. For the case lesion CL, the feature vector is calculated in advance by the same structure as the feature amount calculation unit 62 and is then stored in the feature amount DB 23B.

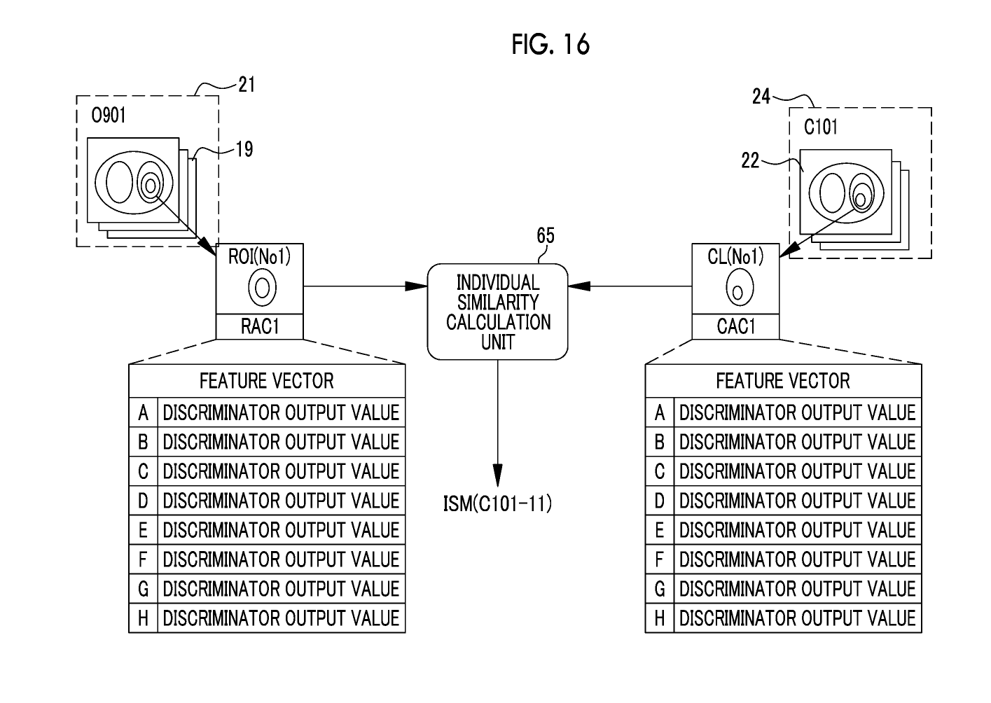

As illustrated in FIG. 16, the individual similarity calculation unit 65 compares the feature amount RAC of the region of interest ROI with the feature amount CAC of the case lesion CL and calculates an individual similarity ISM. Specifically, the individual similarity calculation unit 65 compares the eight-dimensional feature vectors included in the feature amount RAC and the feature amount CAC and calculates the individual similarity ISM. The value of the individual similarity ISM is calculated by, for example, a least square distance or correlation. In the former case, as the value decreases, the similarity between the region of interest ROI and the case lesion CL increases. In the latter case, as the value increases, the similarity between the region of interest ROI and the case lesion CL increases.

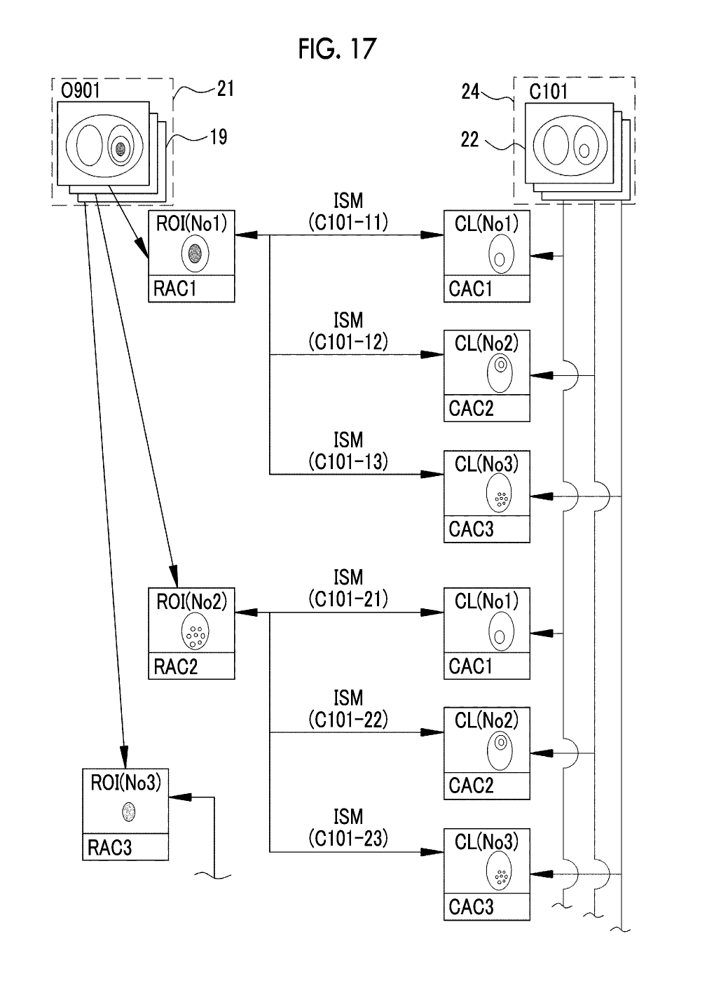

As illustrated in FIG. 17, the individual similarity calculation unit 65 sets a plurality of regions of interest ROI included in one examination data item 21 and a plurality of case lesions CL included in one case data item 24 so as to be in one-to-one correspondence with each other, compares each feature amount RAC with each feature amount CAC, and calculates the individual similarities ISM. Since the individual similarity ISM is an individual similarity for each case lesion CL, it is an individual similarity for each case lesion. In the examination data 21 with the examination ID “O901”, three regions of interest ROI with No1 to No3 are designated. Three case lesions CL with No1 to No3 are registered in the case data 24 with the case ID “C101”. Therefore, in the example illustrated in FIG. 17, a total of nine (=3×3) individual similarities ISM are calculated between the examination data 21 with the examination ID “O901” and the case data 24 with the case ID “C101”. In FIG. 17, the correspondence relationship between the regions of interest ROI with No1 and No2 and the case lesions CL with No1 to No3 is illustrated, but the correspondence relationship between the region of interest ROI with No3 and each case lesion CL is not illustrated due to space restrictions.

An identification code in parentheses which follow each individual similarity ISM is obtained by adding the serial number of each of the region of interest ROI and the case lesion CL to the case ID. For example, “C101-11” indicates an individual similarity ISM between the region of interest ROI with Not and the case lesion CL with Not which is registered in the case data 24 with the case ID “C101”. Similarly, “C101-12” indicates an individual similarity ISM between the region of interest ROI with Not and the case lesion CL with No2 which is registered in the case data 24 with the case ID “C101”.

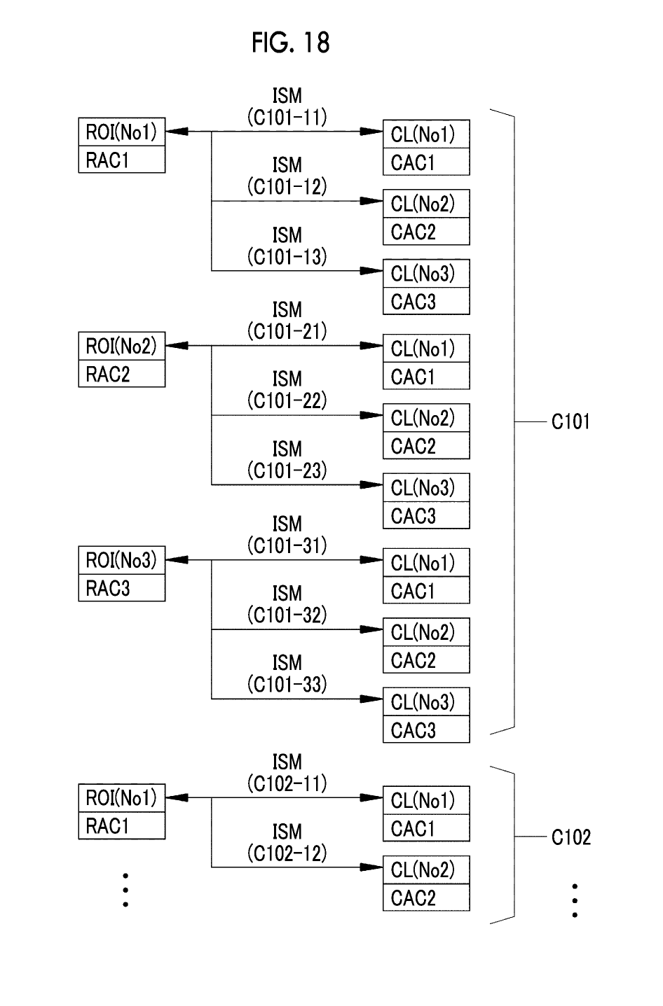

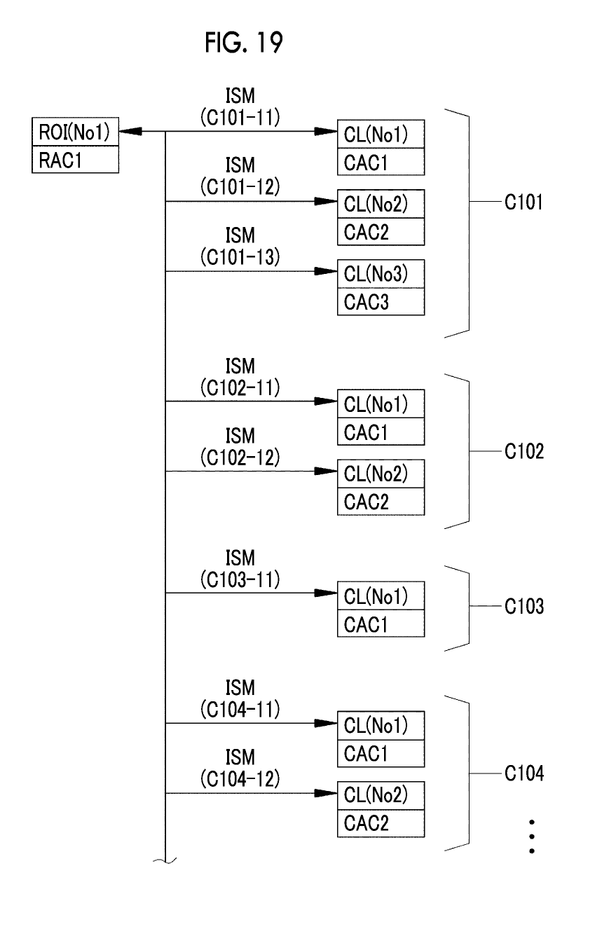

As illustrated in FIGS. 18 and 19, the individual similarity calculation unit 65 calculates the individual similarity ISM for a plurality of cases. For example, as illustrated in FIG. 18, first, the individual similarity calculation unit 65 sets three case lesions CL with No1 to No3 in one case with the case ID “C101” so as to correspond to the regions of interest ROI with No1 to No3, respectively, and calculates the individual similarity ISM. As described above, three case lesions CL (No1 to No3) are registered in the case with the case ID “C101”. Therefore, in the event that three case lesions CL correspond to three regions of interest ROI, a total of nine (=3×3) individual similarities ISM are calculated.

In the event that a process of calculating the individual similarity ISM for the case with the case ID “C101” ends, the individual similarity calculation unit 65 calculates the individual similarity ISM for one case with a case ID “C102”. Two case lesions CL (No1 and No2) are registered in the case with the case ID “C102”. Therefore, in the event that two case lesions CL correspond to three regions of interest ROI, a total of six (=3×2) individual similarities ISM are calculated. This process is repeatedly performed the number of times corresponding to the number of cases.

In FIG. 18, the correspondence relationship between three regions of interest ROI with No1 to No3 and all of the case lesions CL of the case with the case ID “C101” is illustrated. However, for the case with the case ID “C102”, only the correspondence relationship between the region of interest ROI with Not and the case lesions CL is illustrated and the correspondence relationship between the regions of interest ROI with No2 and No3 and the case lesions CL is not illustrated in FIG. 18.

Similarly, the individual similarity calculation unit 65 sets the regions of interest ROI with Not to No3 and the case lesions CL of a case with a case ID “C103” and the subsequent cases so as to correspond to each other and calculates the individual similarities ISM. FIG. 19 illustrates the correspondence relationship between the region of interest ROI with No1 and the case lesions CL of the cases with case IDs “C101” to “C104”. Cases after the case with the case ID “C104” are not illustrated. The individual similarities ISM between the regions of interest ROI with No2 and No3 and the case lesions CL are calculated, which is not illustrated in FIG. 19.

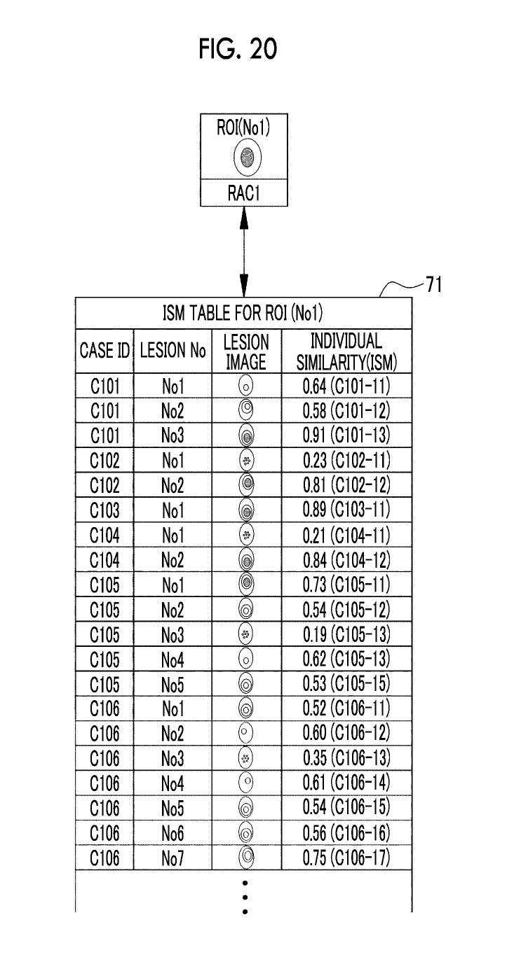

As illustrated in FIG. 20, for example, the individual similarity calculation unit 65 creates an individual similarity table (hereinafter, referred to as an ISM table) 71 in the memory 42B or the storage device 43B of the similar case search server 17 and registers the calculated individual similarities ISM in the ISM table 71. The ISM table 71 is created for each region of interest ROI. In the example illustrated in FIG. 20, the ISM table 71 for the region of interest ROI with Not is illustrated. The ISM table 71 is a table in which a case ID, a lesion number, and a lesion image are stored so as to be associated with each individual similarity ISM. The lesion image is image data of the case lesion CL. That is, in the ISM table 71, one record includes four data items, that is, the case ID, the lesion number, the lesion image, and the individual similarity ISM.

First, the individual similarity calculation unit 65 records each individual similarity ISM in the ISM table 71 in a calculation order. The individual similarities ISM are recorded in ascending order of the number of the case ID, such as in the order of “C101”, “C102”, and “C103”. The value of the individual similarity ISM is calculated by the correlation between the feature amount RAC of the region of interest ROI and the feature amount CAC of the case lesion CL. Therefore, as the value increases, the similarity increases.

Similarly to the above-mentioned feature vector, the individual similarity ISM may be calculated by a least square distance. In this case, as the value of the least square distance decreases, the similarity increases.

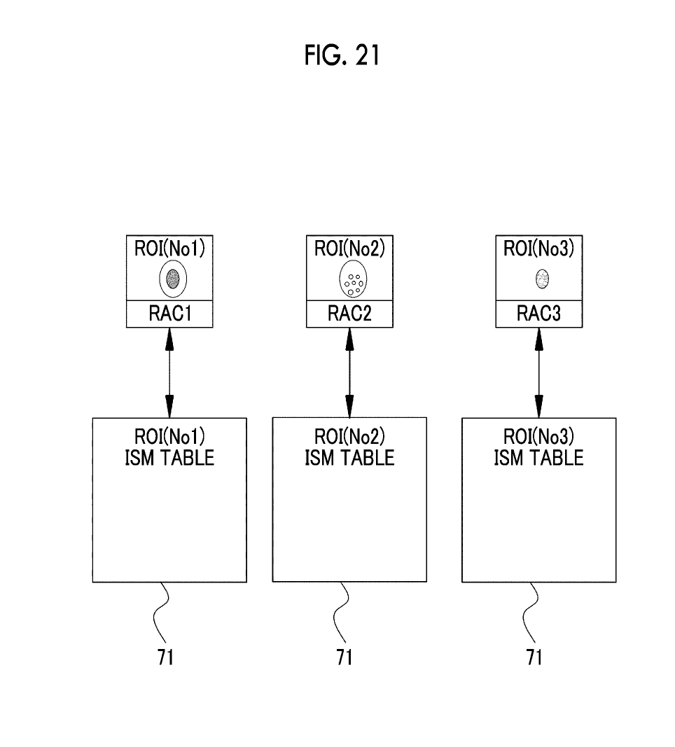

As illustrated in FIG. 21, the individual similarity calculation unit 65 creates the ISM table 71 for each region of interest ROI. In a case in which there are three regions of interest ROI with No1 to No3, three ISM tables 71 are created. In this stage, as illustrated in FIG. 20, in the ISM tables 71, each record is arranged in the order of the number of the case ID. In a case in which the creation of the ISM tables 71 ends, the individual similarity calculation unit 65 transmits the ISM tables 71 to the similar case search unit 67.

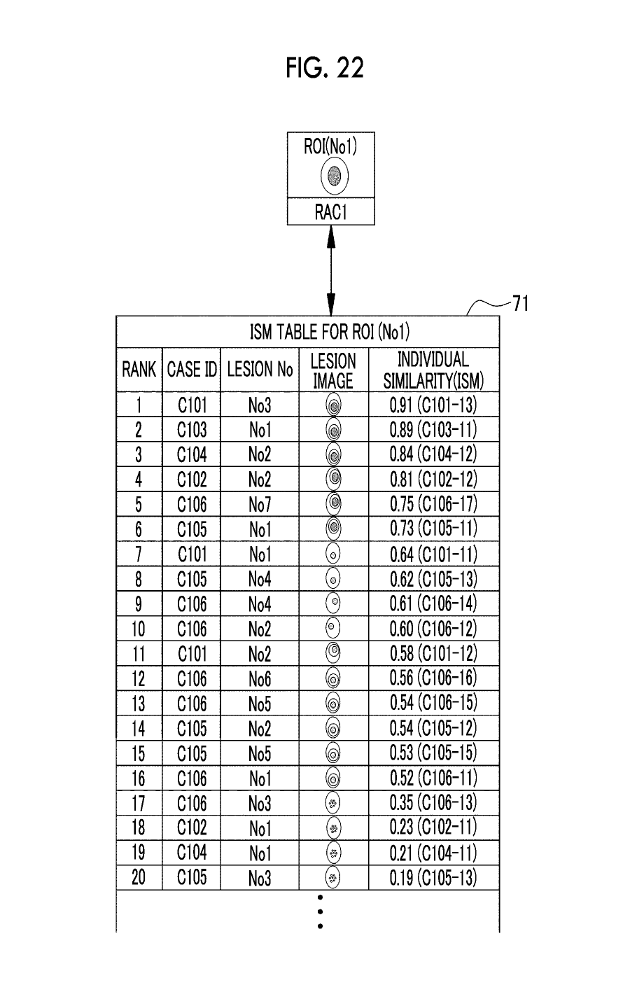

As illustrated in FIG. 22, the similar case search unit 67 sorts records in descending order of the individual similarity ISM in each ISM table 71. In this way, the case lesions CL are ranked. Then, the case lesions CL are extracted such that a case lesion CL with a higher similarity to the region of interest ROI is ranked higher in the ISM table 71.

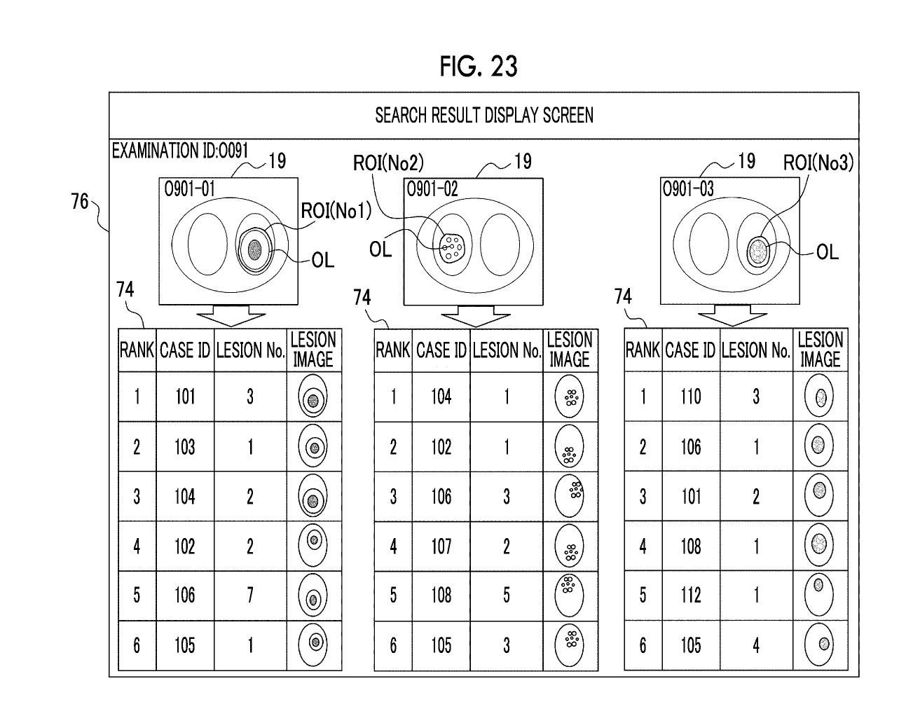

The similar case search unit 67 is provided with a list creation unit 67A (see FIG. 11). The list creation unit 67A creates a similar case list 74 illustrated in FIG. 23 on the basis of the ISM table 71. The similar case list 74 is displayed on a search result display screen 76. The similar case list 74 is a list of information related to a plurality of similar cases. Specifically, the similar case list 74 is a similar case list for each region of interest which corresponds to each ISM table 71 for each region of interest ROI. The search result display screen 76 is an example of a screen that is transmitted as the search result from the similar case search server 17 to the treatment department terminal 11 which is the request source of the similar case search request.

The list creation unit 67A extracts a rank, a case ID, a lesion No, and a lesion image for one case lesion CL from each ISM table 71 and arranges the extracted items in descending order of the individual similarity ISM to create a similar case list 74. For example, the top six case lesions CL in the similar case list 74 are displayed. Of course, the case lesions CL in sixth place or lower may be displayed by, for example, a screen scroll operation. In addition, the number of case lesions which can be displayed at the same time may be changed such that the top ten case lesions are displayed.

Since the similar case list 74 is extracted for each region of interest ROI, the examination image 19 including a corresponding region of interest ROI ranks high in each list 74 on the search result display screen 76. In this case, it is possible to compares the similar case list 74 and the corresponding region of interest ROI, which makes it easy to intuitively determine the similarity between the region of interest ROI and the case lesion CL.

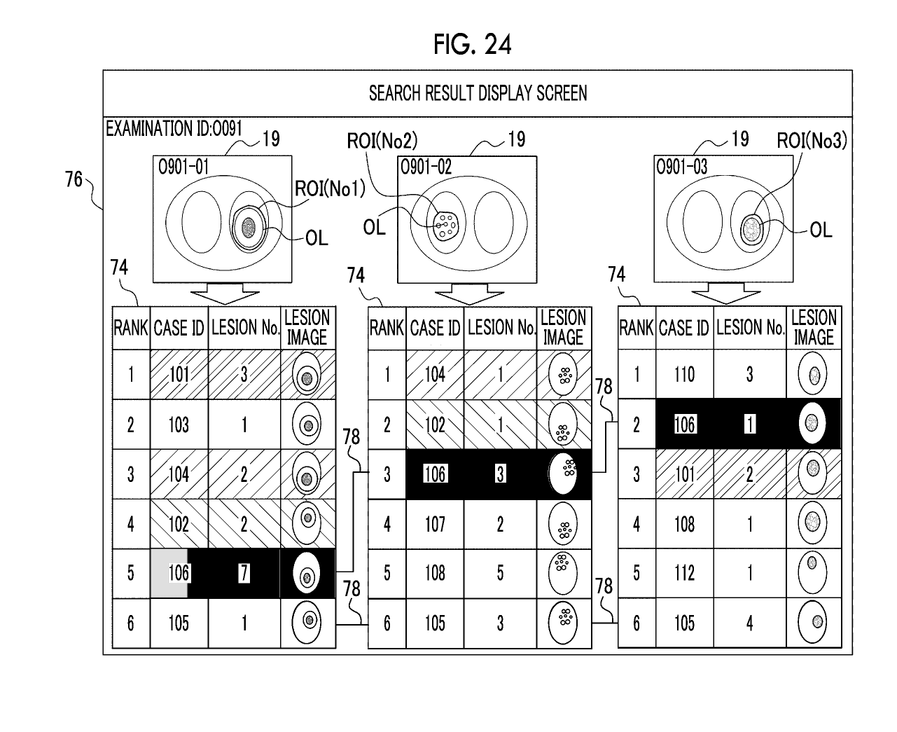

As illustrated in FIG. 24, in a case in which there are case lesions CL included in a common case in each similar case list 74, the list creation unit 67A displays a plurality of case lesions CL in a common case so as to be identified. For example, in each of the similar case lists 74 corresponding to the regions of interest ROI with Not to No3, there are case lesions CL (No7, No3, and Not) included in a case with a case ID “C106”. In addition, in each similar case list 74, similarly, there are case lesions CL (No1, No3, and No4) included in a case with a case ID “C105”.

As such, in each similar case list 74, there are case lesions CL included in a common case. In this case, the list creation unit 67A displays a plurality of case lesions CL included in a common case so as to be identified by, for example, a method that displays the case lesions to have the same background color or a method that connects the case lesions CL with a connection line 78. In FIG. 24, thin black lines with the same density and hatched portions of the same type indicate the case lesions with the same background color. In this way, it is possible to check the case lesions CL included in a common case at a glance in each similar case list 74.

In addition to the cases in which the case lesions CL are present in all of three similar case lists 74 like the case with the case ID “C106” or “C105”, for example, in a case in which the case lesions CL included in a common case are present in two lists, that is, the similar case list 74 corresponding to the region of interest ROI with Nol and the similar case list 74 corresponding to the region of interest ROI with No3, like the case lesions CL included in the case with the case ID “C101”, the list creation unit 67A displays the case lesions CL so as to be identified.

The output control unit 69 performs control such that extensible markup language (XML) data for web distribution is created for the created search result display screen 76 by a markup language, such as XML, and is transmitted as the search result to the treatment department terminal 11 which is a request source. In the treatment department terminal 11 which has received the XML data, a web browser reproduces the search result display screen 76 on the basis of the XML data and displays the search result display screen 76 on the display unit 48A. In this way, the doctor browses the search result display screen 76 including the similar case list 74.

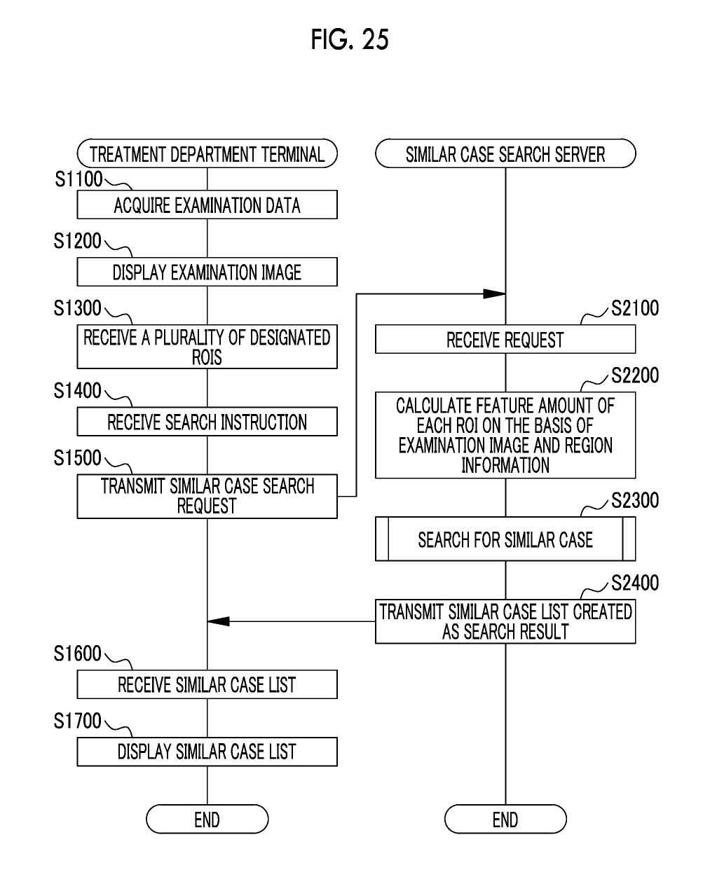

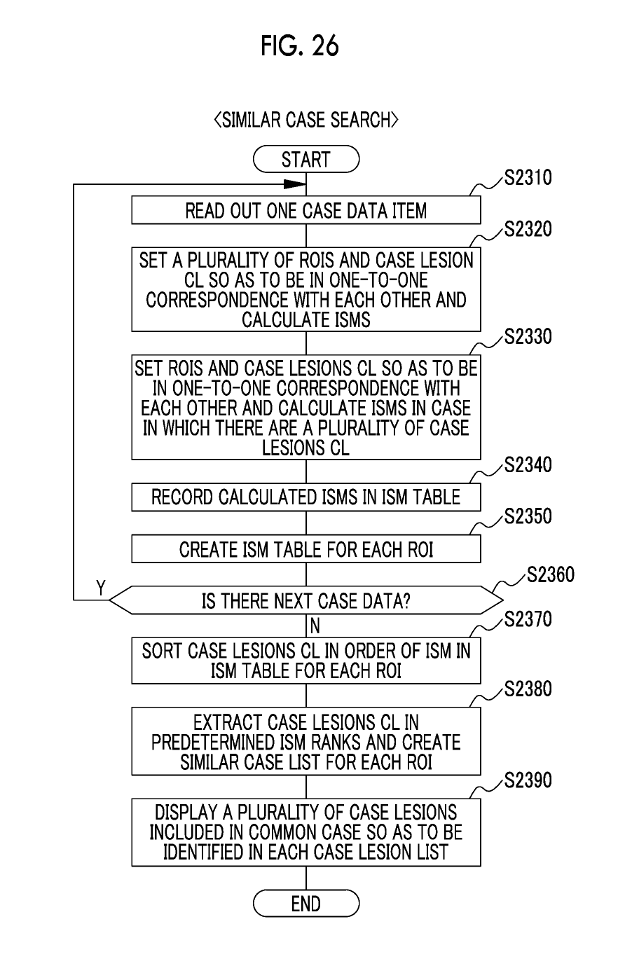

Next, the operation of the above-mentioned structure will be described with reference to FIGS. 25 and 26. As illustrated in FIG. 25, the doctor in the treatment department 10 accesses the examination image DB server 15, using the treatment department terminal 11, and acquires the examination data 21 of the examination requested to the examination department 12 (S1100). The treatment department terminal 11 displays the examination data 21 on the display unit 48A (S1200). The examination images 19 included in the acquired examination data 21 are displayed on the examination image display screen 52 illustrated in FIG. 9. The doctor designates the regions of interest ROI in the examination images 19 through the examination image display screen 52. The treatment department terminal 11 receives a plurality of regions of interest ROI designated by the designation operation of the doctor (S1300). In the event that the designation of the regions of interest ROI ends, the similar case search button 52E is operated. Then, the treatment department terminal 11 receives a search instruction (S1400). In the event that the search instruction is received, the search request issuing unit 54 issues a similar case search request to which the examination images 19 and region information are added and transmits the similar case search request to the similar case search server 17 (S1500).

In the event that the similar case search server 17 receives the similar case search request, the request receiving unit 61 receives the similar case search request (S2100). Then, the feature amount calculation unit 62 calculates the feature amount of each region of interest ROI on the basis of the examination images 19 and the region information of the regions of interest ROI (S2200). Then, a similar case search process is performed (S2300).

As illustrated in FIG. 26, in the similar case search process, first, the individual similarity calculation unit 65 reads out one case data item 24 from the case DB server 16 (S2310). Then, the individual similarity calculation unit 65 calculates the individual similarities ISM using the one-to-one correspondence between a plurality of regions of interest ROI in the examination data 21 and the case lesions CL included in one case data item 24 (S2320). In a case in which there are a plurality of case lesions CL, the individual similarity ISM is calculated for each case lesion CL (S2330). The individual similarity calculation unit 65 records the calculated individual similarities ISM in the ISM table 71 (S2340) and creates the ISM table 71 for each region of interest ROI (S2350). After this process is performed for one case data item 24, it is performed for the next case data 24 using the same method as described above. Then, the same process is repeatedly performed until the calculation of the individual similarity ISM and the creation of the ISM table 71 for all of the case data 24 end (N in S2360).

The similar case search unit 67 searches for similar cases on the basis of the ISM tables 71 created for each region of interest ROI. First, the similar case search unit 67 sorts the case lesions CL in descending order of the individual similarity ISM in each ISM table 71 (S2370). In this way, a case lesion CL with a higher similarity to the region of interest ROI is extracted and ranked higher in each ISM table 71.

The list creation unit 67A extracts the case lesions CL up to a threshold rank (sixth in this example) from each of the sorted ISM tables 71 and creates the similar case list 74. Then, the list creation unit 67A creates the search result display screen 76 (see FIG. 23) including the similar case lists 74 for each region of interest ROI and the examination images 19 including the regions of interest ROI corresponding to each similar case list 74 (S2380). In addition, in a case in which there are a plurality of case lesions CL in a common case in the similar case list 74, the list creation unit 67A displays the case lesions CL so as to be identified by, for example, a method that displays the case lesions CL so as to have the same background color (S2390).

The output control unit 69 converts the search result display screen 76 including the similar case list 74 which has been created as the search result by the list creation unit 67A into XML data for distribution and transmits the XML data to the treatment department terminal 11 (S2400). The treatment department terminal 11 receives the XML data including the similar case list 74 (S1600), reproduces the search result display screen 76 (see FIG. 24) on the basis of the XML data, and displays the search result display screen 76 on the display unit 48A.

The similar case list 74 is created on the basis of the individual similarities ISM that is calculated by the one-to-one correspondence between a plurality of regions of interest ROI, each of which includes one or more different target lesions OL, and a plurality of case lesions CL. Therefore, in a case in which a plurality of regions of interest ROI are designated in one examination data item 21 including one or more examination images 19, it is possible to search for a similar case including a case lesion CL, while paying attention to the feature amount of each region of interest ROI.

In the related art which searches for a similar case while paying attention to only the feature amount of one region of interest ROI, in a case in which it is necessary to pay attention to a plurality of regions of interest ROI, the number of searches increases depending on the number of regions of interest ROI such that, after one region of interest ROI is designated and a search is performed, another region of interest ROI is designated and a search is performed. In contrast, in the invention, a plurality of designated regions of interest ROI are received and it is possible to perform a similar case search in which attention is paid to the feature amount of each region of interest ROI. Therefore, the time and effort required to perform a search are reduced. In addition, even though a similar case search process based on one region of interest ROI is performed a plurality of times, a plurality of search results are individual presented. Therefore, it is difficult to compare and determine the search results. In contrast, as in the invention, in the event that a similar case search process based on a plurality of regions of interest ROI is performed, it is possible to present the search results related to a plurality of regions of interest ROI in the form in which the search results are easily compared, like the search result display screen 76 illustrated in FIG. 24.

In some cases, a disease is specified on the basis of whether a plurality of target lesions OL appear. As such, the invention is useful to diagnose a disease in which attention needs to be paid to the feature amounts of a plurality of regions of interest ROI. In many cases, this disease is a non-cancerous disease, such as tuberculosis in which attention needs to be paid to three types of target lesions OL, that is, a vomica shadow (cavity), a punctate shadow (small nodules), and a frosted glass shadow (ground glass opacity), or diffuse panbronchiolitis in which attention needs to be paid to two types of target lesions OL, that is, an abnormal shadow of the bronchus and a punctate shadow. Therefore, the invention is particularly useful for the diagnosis of a non-cancerous disease.

The search result is displayed in the form of the similar case list 74 which is a list of information related to a plurality of similar cases, which makes it easy to check similar cases. In addition, since the similar case list 74 is created for each region of interest ROI, it is easy to check which case lesions CL are similar to each region of interest ROI.

In a case in which there are case lesions CL included in a common case in each similar case list 74, the case lesions CL are displayed so as to be identified by, for example, a method that displays the case lesions to have the same background color or a method that connects the case lesions CL with the connection line 78. Therefore, it is possible to check the case lesions CL in a common case at a glance. As described above, a non-cancerous disease is specified on the basis of whether a plurality of target lesions OL appear. In the diagnosis, a case including a plurality of case lesions CL which are similar to a plurality of regions of interest ROI in one examination data item 21 is first referred to. Therefore, in each similar case list 74, displaying the case lesions CL included in a common case so as to be identified is particularly useful for a diagnosis for a non-cancerous disease in which attention needs to be paid to a plurality of regions of interest ROI.

Since the case lesions CL included in a common case are displayed so as to be identified, the doctor can use the case lesions CL while determining whether to make a diagnosis on the basis of a case with a high individual similarity or to make a diagnosis on the basis of a case with a high total similarity, according to the situation, which is convenient.

The identification display indicating that there is a common case makes it easy to compare two cases including the case lesions CL that are present in three similar case lists 74, such as cases with the case IDs “C106” and “C105” described in FIG. 24. For example, as can be seen from FIG. 24, three case lesions CL in the case with the case ID “C106” rank higher than three case lesions CL in the case with the case ID “C105” in each similar case list 74. Therefore, in the event that the cases with the cases ID “C106” and “C105” are compared, it is possible to easily determine that the case with the case ID “C106” is more appropriate as a similar case than the case with the case ID “C105”.

In this example, the target lesions OL of different types, such as “vomica” and “a frosted glass shadow”, are included in a plurality of regions of interest ROI. However, the target lesions OL included in each region of interest ROI may be the same type as long as they are different from each other.

### Second Embodiment

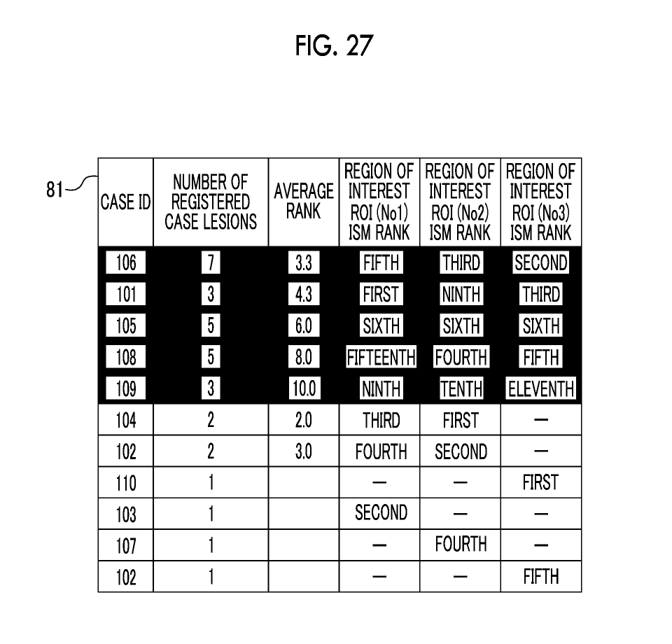

An average rank list 81 illustrated in FIG. 27 may be transmitted as the search result, in addition to the similar case list 74 or instead of the similar case list 74. The average rank list 81 is used to easily check a similar case that is most similar to the examination data 21 among a plurality of cases in which the number of registered case lesions CL is equal to or greater than the number of regions of interest ROI designated in the examination data 21. The average rank list 81 is created by the list creation unit 67A.

In the average rank list 81, a plurality of cases are arranged, using two items, that is, the number of case lesions CL registered in one case and an average rank, as sort keys. Of two sort keys, the number of registered case lesions CL has a higher priority than the other. The cases are arranged in descending order of the number of registered case lesions CL, using the number of registered case lesions CL as a sort key. In this stage, a case with a case ID “C106” includes seven registered case lesions which are the largest number of registered case lesions, followed by cases with case IDs “C105” and “C108” including five registered case lesions. Then, cases with case IDs “C101” and “C109” including three registered case lesions follow the cases with the case IDs “C105” and “C108”.

The average rank is the rank of the average value of the ranks of individual similarities ISM for each region of interest ROI (No1 to No3). The ranks of the individual similarities ISM are the ranks of the similar case lists 74. Since the case with the case ID “C106” is ranked fifth, third, and second in three similar case lists 74, the average rank is 3.3 (=(5+3+2)/3). Similarly, the average rank of the case with the case ID “C101” is 4.3 and the average rank of the case with the case ID “C105” is 6.0. The list creation unit 67A sorts the cases (including three or more case lesions CL) in which the number of registered case lesions CL is equal to or greater than the number of regions of interest ROI (three regions of interest ROI in this example) in the order of the average rank.

A case having a high average rank means that a case includes the case lesions CL having a high average similarity with respect to each region of interest ROI. From one point of view, the case including the case lesions CL having a high average similarity can be evaluated to be a similar case that is most similar to the examination data 21. Therefore, the average rank list 81 makes it possible to simply check a similar case that is most similar to the examination data 21 among a plurality of cases in which the number of registered case lesions CL is equal to or greater than the number of regions of interest ROI.

Among the cases in which the number of registered case lesions CL is less than the number of regions of interest ROI (three regions of interest ROI), the average rank of a case in which the number of registered case lesions CL is equal to or greater than 2 is calculated. However, the case in which the number of registered case lesions CL is less than the number of regions of interest ROI is evaluated to have a low similarity in terms of numbers. Therefore, even assuming that the average rank of the case is high, the case is displayed at a lower rank than the case in which the number of registered case lesions CL is equal to or greater than the number of regions of interest ROI. For the case including one registered case lesion, the calculation of the average rank of the case is meaningless and the average rank of the case is not calculated.

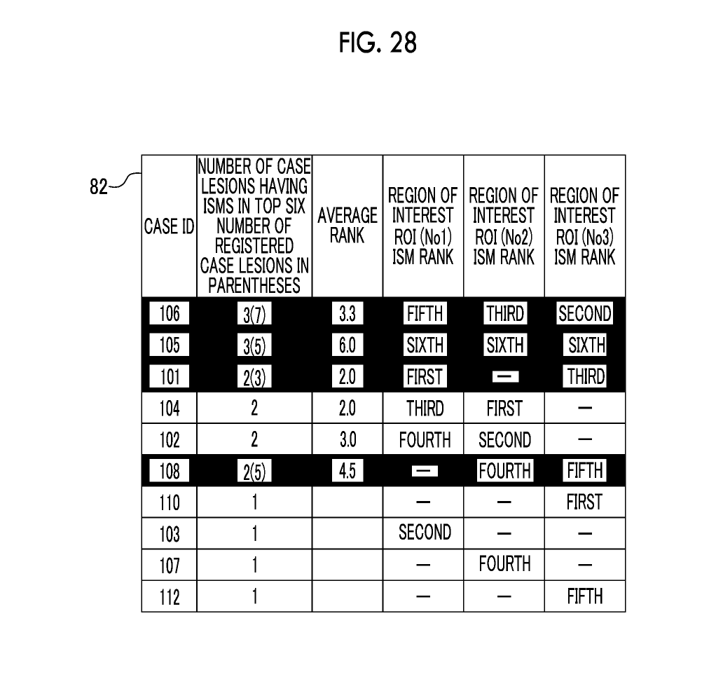

Instead of the average rank list 81 illustrated in FIG. 27, an average rank list 82 illustrated in FIG. 28 may be created. The average rank list 82 differs from the average rank list 81 in that the ranks of the individual similarities ISM of each similar case list 74, of which the average rank is to be calculated, are limited to the top six. Since the cases with the case IDs “C106” and “C105” include three case lesions CL that are ranked in top six, the average rank of the case lesions CL is calculated. In the case with the case ID “C101”, the number of registered case lesions CL is 3, but the number of case lesions CL which are ranked in the top six is 2. Therefore, the average rank of only the two case lesions CL is calculated. The average rank list 82 makes it easy for the user to see the case lesions CL with a high average similarity.

### Third Embodiment

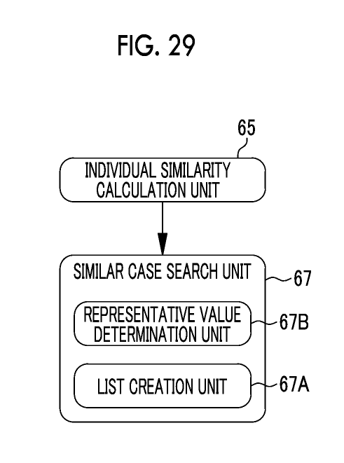

In a third embodiment illustrated in FIGS. 29 to 32, in the event that an ISM table 71 is created for one region of interest ROI, one representative value is determined for each case. As illustrated in FIG. 29, in the third embodiment, a similar case search unit 67 is provided with a representative value determination unit 67B.

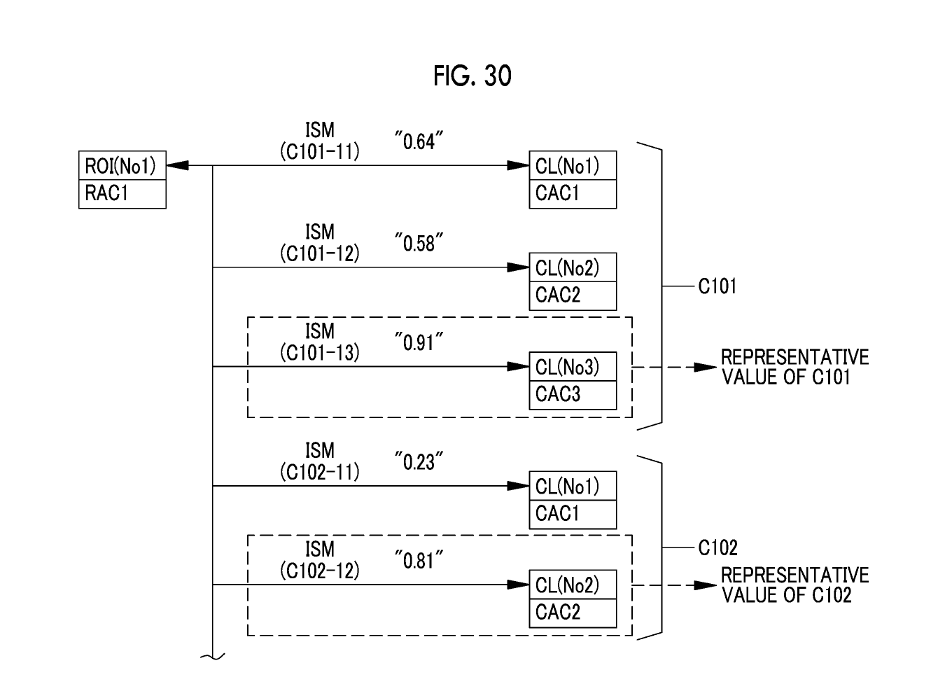

As illustrated in FIG. 30, the individual similarity calculation unit 65 calculates the individual similarities ISM between each case lesion CL and one region of interest ROI. The representative value determination unit 67B determines a representative value from the individual similarities ISM for each case lesion CL. In this example, in a case with a case ID “C101”, the individual similarity ISM between a case lesion CL with No3 and a region of interest ROI with Not is the highest. Therefore, the case lesion CL with No3 is a representative case lesion of the case with the case ID “C101”. Similarly, a case lesion CL with No2 is a representative case lesion of a case with a case ID “C102”.

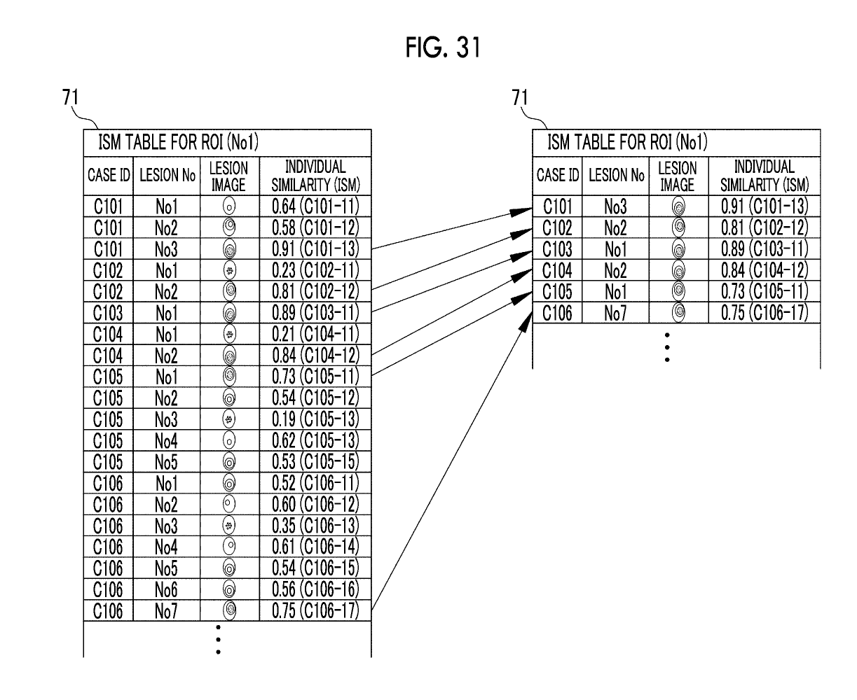

As illustrated in FIG. 31, the representative value determination unit 67B performs the representative value determination process for all of the cases. In this way, one case lesion CL is extracted for one case from the ISM table 71 for one region of interest ROI and it is possible to reduce the number of case lesions CL, as compared to the first embodiment. Therefore, it is possible to reduce the processing load of the similar case search unit 67 and thus to perform a search process at a high speed.

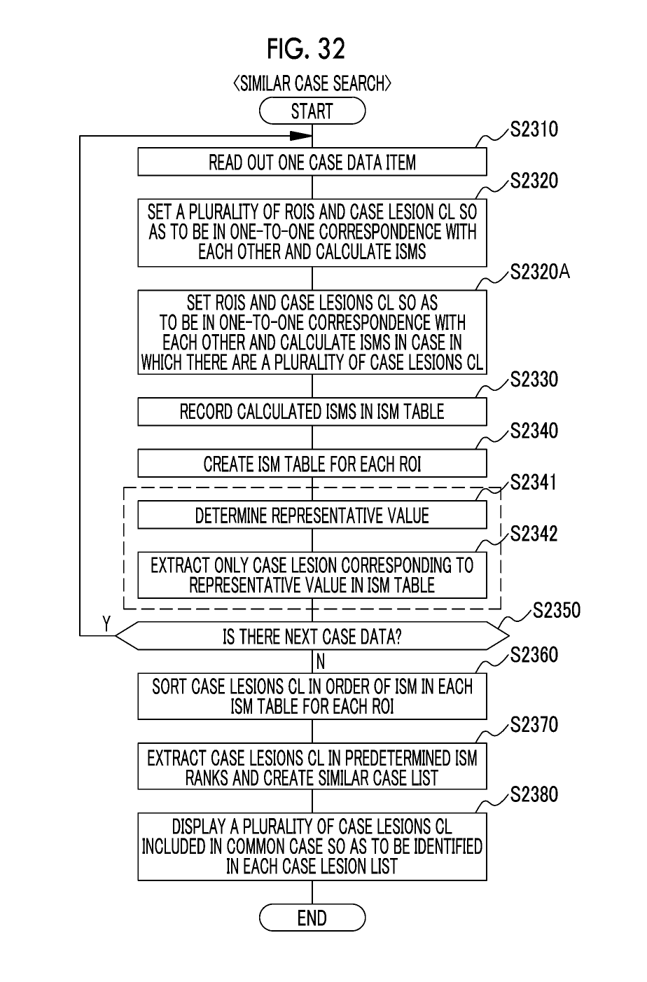

As illustrated in FIG. 32, for example, the individual similarity calculation unit 65 creates the ISM table 71 for each region of interest ROI (S2340). Then, the representative value determination unit 67B determines a representative value for each case (S2341) and extracts only the case lesion CL corresponding to the representative value in the ISM table 71 (S2342). The subsequent steps are the same as those in the first embodiment and thus the description thereof will not be repeated. After the individual similarities ISM for the case lesions CL in all of the cases are calculated and the representative values for each case are determined in the ISM table 71. However, for example, the individual similarity calculation unit 65 may perform the representative value determined process whenever the individual similarities ISM for the case lesions CL in one case are calculated. In this case, it is possible to reduce a work area of a memory in which the ISM table 71 is temporarily developed.

### Fourth Embodiment

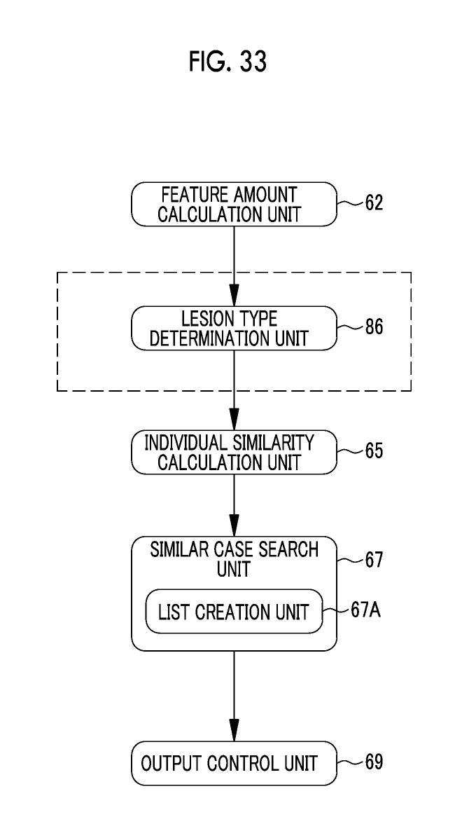

As illustrated in FIGS. 33 to 36, individual similarities ISM may be calculated by only the correspondence between a target lesion OL included in a region of interest ROI and a case lesion CL which are the same type. As illustrated in FIG. 13, lesion patterns are typically distinguished by the type of lesion. Therefore, in a stage in which a feature amount is calculated, it is possible to determine the type of lesion on the basis of the feature amount. In a fourth embodiment, the determination of the type of lesion is used.

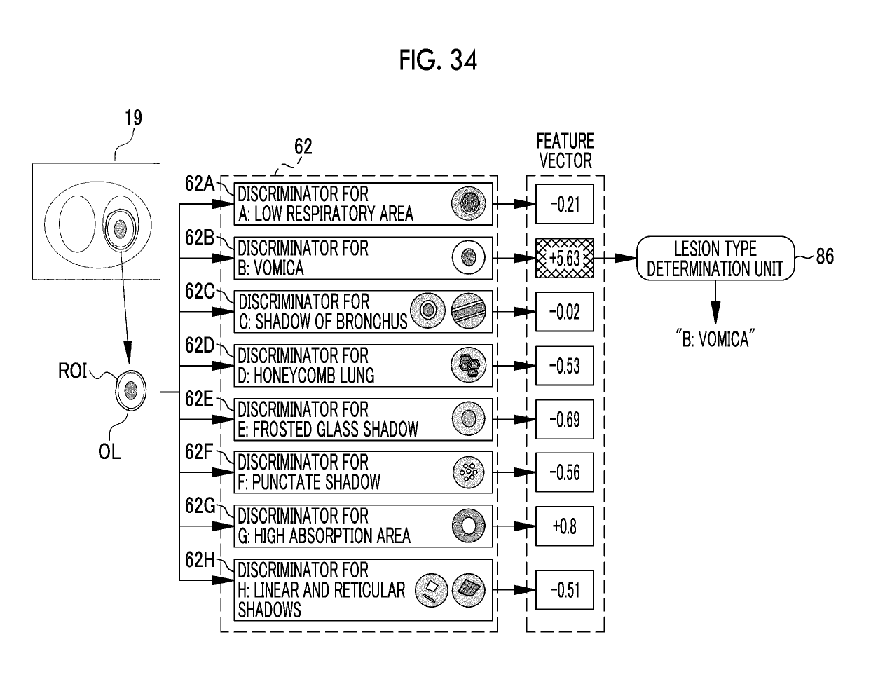

As illustrated in FIG. 33, in the fourth embodiment, a similar case search server 17 is provided with a lesion type determination unit 86. As illustrated in FIG. 34, the lesion type determination unit 86 determines the type of target lesion OL included in the region of interest ROI on the basis of the feature amount RAC of the region of interest ROI calculated by a feature amount calculation unit 62. For example, the lesion type determination unit 86 determines the type of lesion corresponding to a discriminator indicating the maximum discriminator output value among the discriminator output values from discriminators 62A to 62H to be the type of target lesion OL included in the region of interest ROI. In this example, since the discriminator output value from the discriminator 62B corresponding to “B: vomica” is the maximum, the type of target lesion OL is determined to be “B: vomica”.

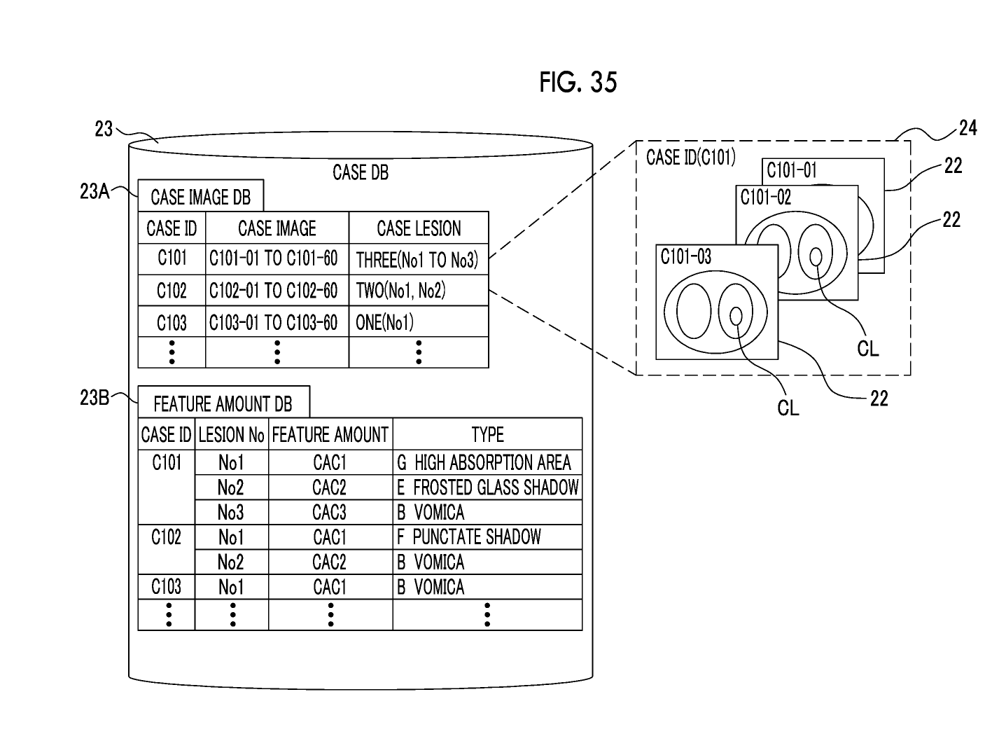

In the fourth embodiment, as illustrated in FIG. 35, for each case lesion CL, the type of lesion is determined in advance on the basis of the feature amount CAC and the determined type of lesion is stored in a feature amount DB 23B.

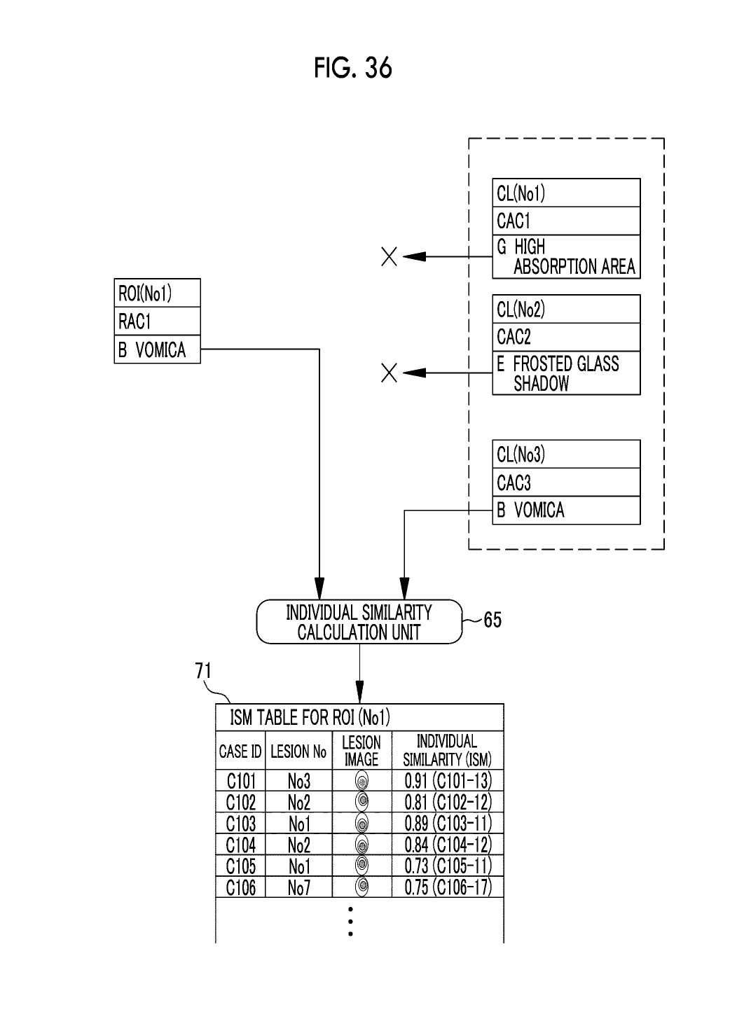

As illustrated in FIG. 36, in the event of calculating the individual similarities ISM between the region of interest ROI and each case lesion CL, the individual similarity calculation unit 65 calculates the individual similarity ISM between the lesions of the same type and does not calculate the individual similarity ISM between different types of lesions. In this example, since a region of interest ROI with No1 is the type “B: vomica”, the individual similarity calculation unit 65 calculates only the individual similarity ISM between the region of interest ROI and a case lesion CL with No3, of which the type is “B: vomica”, in a case with a case ID “C101”. In a case in which a plurality of case lesions CL which are the same type as the region of interest ROI are registered in one case, a plurality of individual similarities ISM are calculated. In a case in which no case lesion CL which is the same type as the region of interest ROI is registered, the individual similarity ISM is not calculated for the case.

In this way, it is possible to reduce the calculation time of the individual similarity calculation unit 65. In addition, the size of the ISM table 71 is smaller than that in the first embodiment in which the individual similarity ISM is calculated without distinguishing the types of case lesions, which results in a reduction in the work area of a memory. Therefore, load applied to the CPU 41B of the similar case search server 17 is reduced. As a result, it is possible to reduce the search time. The effect of reducing the size of the ISM table 71 is obtained, which is the same as that in the third embodiment illustrated in FIG. 29. In the fourth embodiment, the effect of reducing the search time by a value corresponding to a reduction in the processing time of the individual similarity calculation unit 65 is more remarkable than that in the third embodiment.

However, in the aspect in which the type of lesion is determined in advance and only the individual similarity ISM between the lesions of the same type is calculated, in a case in which the accuracy of determining the type of lesion is low, so-called search omission in which the case lesion CL to be searched as a similar case is missing is likely to occur. In particular, as illustrated in FIG. 10, in a case in which a plurality of target lesions OL are designated as one region of interest ROI, the type of lesion is determined on the basis of only one of a plurality of target lesions OL. For this reason, it is preferable that the fourth embodiment is performed after the accuracy of determining the type of lesion is checked.

### Fifth Embodiment

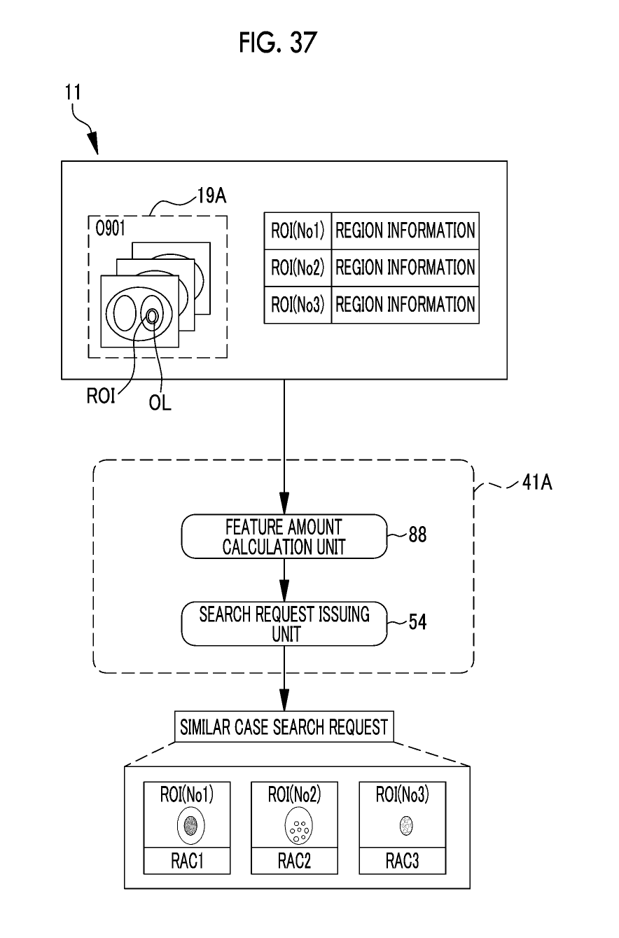

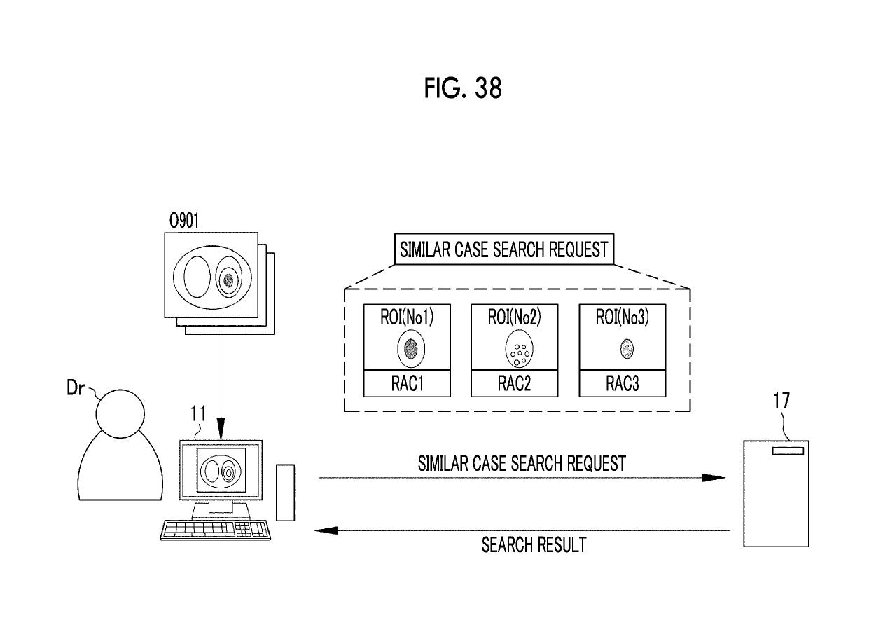

In a fifth embodiment illustrated in FIGS. 37 and 38, not the similar case search server 17 but the treatment department terminal 11 calculates the feature amount of the region of interest ROI. As in the fifth embodiment, the treatment department terminal 11 may calculate the feature amount of the region of interest ROI. In this case, the similar case search server 17 does not include the feature amount calculation unit and includes structures other than the feature amount calculation unit 62, such as the individual similarity calculation unit 65 and the similar case search unit 67 illustrated in FIG. 11.

As illustrated in FIG. 37, the treatment department terminal 11 is provided with a feature amount calculation unit 88 having the same structure as the feature amount calculation unit 62. For example, a CPU 41A executes software that is installed in the treatment department terminal 11 to implement the feature amount calculation unit 88. The feature amount calculation unit 88 calculates a feature amount RAC on the basis of examination data 21 including an examination image 19 and the region information of the region of interest ROI which is input through a GUI control unit 53. A search request issuing unit 54 attaches an image corresponding to the region of interest ROI and the calculated feature amount RAC to a similar case search request and issues the similar case search request.

As illustrated in FIG. 38, the similar case search request is transmitted from the treatment department terminal 11 to the similar case search server 17. The similar case search server 17 searches similar cases on the basis of the received similar case search request and transmits the search result to the treatment department terminal 11. In the fifth embodiment, in a case in which the lesion determination process described in the fourth embodiment is performed, the treatment department terminal 11 may be provided with a lesion type determination unit. In this case, the determined type information is added to the similar case search request and the similar case search request is transmitted to the similar case search server 17. In the fifth embodiment, the request receiving unit 61 of the similar case search server 17 functions as a feature amount acquisition unit.

In each of the above-described embodiments, the similar case search device according to the invention has been described in the form of the similar case search server 17 that searches for similar cases on the basis of the request from the treatment department terminal 11. However, the similar case search server 17 may not be used and the treatment department terminal 11 may be provided with the similar case search function such that the treatment department terminal 11 accesses the case DB server 16 and searches for similar cases. In this case, the treatment department terminal 11 is the similar case search device.

In each of the above-described embodiments, the similar case search server 17 and the case DB server 16 are provided as individual servers. However, the similar case search server 17 and the case DB server 16 may be integrated into one server. As such, a plurality of functions may be integrated into one server or may be distributed to different servers.

The hardware configuration of the computer system can be modified in various ways. For example, the similar case search server 17 may be formed by a plurality of server computers which are separated as hardware components in order to improve processing capability or reliability. As such, the hardware configuration of the computer system can be appropriately changed depending on required performances, such as processing capability, safety, and reliability. In addition to hardware, a program, such as the case DB 23 or the AP 50, may be duplicated or may be dispersedly stored in a plurality of storage devices in order to ensure safety or reliability.

In each of the above-described embodiments, the similar case search server 17 is used in one medical facility. However, the similar case search server 17 may be used in a plurality of medical facilities.

Specifically, in each of the above-described embodiments, the similar case search server 17 is connected to client terminals that are installed in one medical facility, such as the treatment department terminals 11, through a LAN such that it can communicate with the client terminals and provides application services related to a similar case search on the basis of requests from the client terminals. The similar case search server 17 is connected to the client terminals installed in a plurality of medical facilities through a wide area network (WAN), such as the Internet or a public telecommunication network, such that it can communicate with the client terminals. In this way, the similar case search server 17 can be used in a plurality of medical facilities. Then, the similar case search server 17 receives requests from the client terminals in the plurality of medical facilities and provides application services related to a similar case search to each client terminal.

In this case, the similar case search server 17 may be installed and operated by, for example, a data center different from the medical facilities or by one of the plurality of medical facilities. In a case in which the WAN is used, it is preferable to construct a virtual private network (VPN) or to use a communication protocol with a high security level, such as hypertext transfer protocol secure (HTTPS), considering information security.

The invention is not limited to each of the above-described embodiments and can use various structures, without departing from the scope and spirit of the invention. For example, in this example, CT images, MRI images, and plain X-ray images are given as examples of the examination image. However, the invention may be applied to examination images which are captured by other modalities, such as a mammography system or an endoscope. In addition, the above-mentioned various embodiments or various modification examples may be appropriately combined with each other. The invention is also applied to storage medium that stores the program for implementing the invention, in addition to the program.

What is claimed is:

1. A similar case search device that searches fora similar case which is similar to an examination image used to diagnose a patient from a case database in which a plurality of cases, each of which includes one or more case images, are registered, comprising:
a feature amount acquisition unit that acquires a feature amount of each of a plurality of regions of interest, each of which is designated so as to include one or more different target lesions that are lesions in the examination images, in examination data including one or more examination images;
an individual similarity calculation unit that compares the feature amount of each region of interest with a feature amount of a case lesion, which is a lesion in the case image registered in the case, and calculates an individual similarity for each region of interest; and
a similar case search unit that searches for the similar case on the basis of a plurality of calculated individual similarities.

2. The similar case search device according to claim 1,
wherein the plurality of regions of interest include different types of lesions.

3. The similar case search device according to claim 1,
wherein, in a case in which a plurality of case lesions are registered in one case, the individual similarity calculation unit sets the plurality of regions of interest and the plurality of case lesions so as to be in one-to-one correspondence with each other, compares the feature amounts, and calculates an individual similarity for each case lesion.

4. The similar case search device according to claim 3,
wherein the similar case search unit creates a similar case list which is a list of information related to a plurality of similar cases.

5. The similar case search device according to claim 4,
wherein the similar case list includes a similar case list for each region of interest.

6. The similar case search device according to claim 5,
wherein the similar case list for each region of interest is a list of the plurality of case lesions.

7. The similar case search device according to claim 6,
wherein, in a case in which there are a plurality of case lesions included in a common case in each similar case list for each region of interest, the plurality of case lesions included in the common case are identifiably displayed to indicate that the case lesions are included in the common case.

8. The similar case search device according to claim 6,
wherein the similar case search unit sorts the plurality of case lesions on the basis of the individual similarity in the similar case list.

9. The similar case search device according to claim 1, further comprising:
a representative value determination unit that, in a case in which the individual similarities are calculated between one region of interest and a plurality of case lesions included in one case, determines one representative value from a plurality of individual similarities,
wherein the similar case search unit searches for the similar case for one region of interest, using only the case lesion corresponding to the representative value.

10. The similar case search device according to claim 1,
wherein the individual similarity calculation unit calculates the individual similarity, using a correspondence between one region of interest and a case lesion that is the same type as the region of interest among the case lesions included in one case.

11. The similar case search device according to claim 10, further comprising:
a lesion type determination unit that determines the type of lesion on the basis of the feature amount of the region of interest.

12. A similar case search method that searches for a similar case which is similar to an examination image used to diagnose a patient from a case database in which a plurality of cases, each of which includes one or more case images, are registered, comprising:
a feature amount acquisition step of acquiring a feature amount of each of a plurality of regions of interest, each of which is designated so as to include one or more different target lesions that are lesions in the examination images, in examination data including one or more examination images;
an individual similarity calculation step of comparing the feature amount of each region of interest with a feature amount of a case lesion, which is a lesion in the case image registered in the case, and calculating an individual similarity for each region of interest; and
a similar case search step of searching for the similar case on the basis of a plurality of calculated individual similarities.

13. A non-transitory computer readable medium for storing a computer-executable program enabling execution of computer instructions to perform operations for searching for a similar case which is similar to an examination image used to diagnose a patient from a case database in which a plurality of cases, each of which includes one or more case images, are registered, said operations comprising:
acquiring a feature amount of each of a plurality of regions of interest, each of which is designated so as to include one or more different target lesions that are lesions in the examination images, in examination data including one or more examination images;
comparing the feature amount of each region of interest with a feature amount of a case lesion, which is a lesion in the case image registered in the case, and calculating an individual similarity for each region of interest; and
searching for the similar case on the basis of a plurality of calculated individual similarities.
